(19) United States
(12) Patent Application Publication
(10) Pub. No.: US 2026/0151241 A1
(43) Pub. Date: Jun. 4, 2026

(54) ADJUSTABLE SPINAL DEVICES AND RELATED DEPLOYMENT AND MEASURING INSTRUMENTS

(71) Applicant: Blue Ocean Spine GmbH, Tuttlingen (DE)

(72) Inventors: Guntmar Eisen, Tuttlingen (DE); Markus Salvermoser, Tuttlingen-Möhringen (DE); Jacob Richter, Tuttlingen (DE)

(21) Appl. No.: 19/262,559

(22) Filed: Jul. 8, 2025

Related U.S. Application Data
(60) Provisional application No. 63/668,889, filed on Jul. 9, 2024.

(51) Int. Cl. A61F 2/46; A61F 2/30

(52) U.S. Cl. CPC A61F 2/4611; A61F 2002/30538; A61F 2002/4627; A61F 2002/4658

(57) ABSTRACT

Adjustable spinal devices, instruments for assessing and measuring the dimensions of an intervertebral space, and instruments for deploying the spinal devices are described. The spinal devices may comprise temporary trial devices or permanent spinal implants, and may be configured for placement between two vertebral bodies. A surgical instrument is described which comprises an elongate shaft having a proximal handle and a distal end portion, a first mating feature for securing the distal end portion of the shaft to the device and a second mating feature for engaging a translation member within the spinal fusion device. The instrument further comprises an adjustment device for moving the translation member to adjust an angle between upper and lower endplates of the device. The instrument may further comprise a third mating feature and a second adjustment device for adjusting a height of the device.

### CROSS-REFERENCE TO RELATED APPLICATIONS

This application claims the benefit of U.S. Provisional Application Ser. No. 63/668,889, filed Jul. 9, 2024, the complete disclosure of which is incorporated herein by reference.

### TECHNICAL FIELD

Measurement and deployment instruments are described herein for use with adjustable implantable devices and their trials for stabilizing and/or promoting the fusion of adjacent bony structures. More particularly, the present disclosure provides instruments that can assess the intervertebral space and adjust the height and/or angle of adjustable implantable spinal fusion cages and their trials to accommodate spacing constraints and/or address lordosis.

### BACKGROUND

Implantable spinal devices can be used to treat a variety of spinal disorders, including degenerative disc disease. For example, in one type of spinal disorder, the intervertebral disc has deteriorated or become damaged due to acute injury or trauma, disc disease or simply the natural aging process. The standard treatment today may involve surgical removal of a portion, or all, of the diseased or damaged intervertebral disc in a process known as a partial or total discectomy, respectively. The discectomy is often followed by the insertion of an interbody cage or spacer to stabilize this weakened or damaged spinal region and/or to restore disc height. This cage or spacer serves to reduce or inhibit mobility in the treated area, in order to avoid further progression of the damage and/or to reduce or alleviate pain caused by the damage or injury. Moreover, these types of cages or spacers serve as mechanical or structural scaffolds to restore and maintain normal disc height, and in some cases, can also provide a space for inserting bone graft material to promote bony fusion between the adjacent vertebrae.

One of the current challenges of these types of procedures is the very limited working space afforded the surgeon to manipulate and insert the cage into the intervertebral area to be treated. Access to the intervertebral space requires navigation around retracted adjacent vessels and tissues such as the aorta, vena cava, dura and nerve roots, leaving a very narrow pathway for access. The opening to the intradiscal space itself is also relatively small. Hence, there are physical limitations on the actual size of the cage that can be inserted without significantly disrupting the surrounding tissue or the vertebral bodies themselves.

Further complicating the issue is the fact that the vertebral bodies are not positioned parallel to one another in a normal spine. There is a natural curvature to the spine due to the angular relationship of the vertebral bodies relative to one another. The ideal interbody fusion cage must be able to accommodate this angular relationship of the vertebral bodies, or else the cage will not sit properly when inside the intervertebral space. An improperly fitted cage would either become dislodged or migrate out of position, and lose effectiveness over time, or worse, further damage the already weakened area.

Another challenge with implanting interbody fusion cages is that, in order to insert the cage between the adjacent vertebra, at least a portion, if not all, of the intervertebral disc is removed to make room for the cage. The removal of the entire disc or disc portion disrupts the normal lordotic or kyphotic curvature of the spine. Traditional fusion cages do not attempt to correct this curvature, and over time as the vertebrae settle around the implanted cages, kyphotic deformity results.

During an implantation procedure, the surgeon must choose a fusion cage that fits the target intervertebral space between adjacent vertebrae. If the selected cage is too small in one dimension, e.g., in height or in lordosis, the envisaged stabilization may not be achieved. If the cage is too big in one dimension, additional pressure on adjacent spinal discs can result, which increases the risk for injuries at the respective tissue. Further, a high risk for damaging the osseous structures of adjacent vertebrae occurs. To reduce the risk of these issues, the surgeon can approach the fitting of the cage by consecutively inserting/implanting and removing several cages in ascending order, i.e., by a trial and an error method. However, this relatively tedious method increases the length of the procedure and may increase collateral tissue damage as multiple cages are inserted and removed.

It is therefore desirable to provide instruments for assessing and measuring a disc space so that the surgeon can choose an optimal cage for that space. It is also desirable to provide deployment instruments for implantable spinal devices that have the ability to maintain and restore the normal anatomy of the fused spine segment. It is particularly desirable to provide instruments for interbody cages or spacers that not only have the mechanical strength or structural integrity to restore disc height or vertebral alignment to the spinal segment to be treated, but also can easily pass through the narrow access pathway into the intervertebral space, and accommodate the angular constraints of this space and/or correct the lordotic or kyphotic curvature created by removal of the disc.

### SUMMARY

The present disclosure provides adjustable spinal devices and related instruments for their deployment. The surgical instruments described herein may be used to assess and/or measure the dimensions of a target intervertebral space and to deploy a variety of different temporary trial devices and/or permanent spinal implants. The trial and spinal implants may be configured for placement between two vertebral bodies and may be particularly useful for placement from a posterior approach outside of the facet joint (transforaminal lumbar interbody fusion or TLIF), an anterior lumbar interbody fusion (ALIF), a posterior lumbar interbody fusion (PLIF) and/or lateral lumbar interbody fusion (LLIF).

In one aspect, a surgical instrument for assessing and/or measuring the dimensions of a target intervertebral space is provided. The instrument comprises an elongate shaft having a proximal handle and a distal end portion, a first mating feature for securing the distal end portion of the shaft to a trial device and a second mating feature for engaging a translation member within the trial device. The instrument further comprises an adjustment device for moving the translation member to adjust an angle between upper and lower endplates of the trial device.

In some embodiments, the adjustment device adjusts a distance between the distal ends of the upper and lower endplates of the trial device to adjust the lordosis angle. In other embodiments, the adjustment device adjusts a distance between the lateral sides of the upper and lower endplates to adjust the lordosis angle. In other embodiments, the adjustment device adjusts the overall height of the trial device by moving the upper and lower endplates towards and away from each other.

In some embodiments, the instrument further comprises one or more gauges for measuring the lordosis angle and/or height of the trial device within the intervertebral space. This allows the surgeon to determine the optimal size and shape of the spinal device to be implanted.

In another aspect, a surgical instrument for inserting a medical device between adjacent vertebral bodies in a patient is provided. The instrument comprises an elongate shaft having a proximal handle and a distal end portion, a first mating feature for securing the distal end portion of the shaft to the device, and a second mating feature for engaging a translation member within the medical device. The instrument further comprises an adjustment device for moving the translation member to adjust an angle between upper and lower endplates of the medical device. The medical device may comprise a trial device or a spinal implant.

In various embodiments, the instrument further comprises an actuator rod extending through the shaft and coupling the adjustment device with the second mating feature. The adjustment device comprises a rotatable handle and a shaft. The shaft is configured for insertion into a proximal opening on the rotatable handle of the adjustment device. Rotation of the handle of the adjustment device causes longitudinal translation of the actuator rod. In some embodiments, the rod is translated proximally to increase this distance and in other embodiments, the rod is translated distally to increase this distance. Translation of the rod, in turn, adjusts a lordosis angle or anterior height of the upper and lower endplates of the medical device.

In some embodiments, the adjustment device adjusts a distance between the distal ends of the upper and lower endplates to adjust the lordosis angle. In other embodiments, the adjustment device adjusts a distance between the lateral sides of the upper and lower endplates to adjust the lordosis angle.

In various embodiments, the instrument further comprises a gauge on the proximal handle. The gauge is configured to indicate the lordosis angle between the upper and lower endplates of the spinal device. In an exemplary embodiment, the gauge comprises a needle and is coupled to the shaft of the adjustment device such that rotation of said shaft causes rotation of the needle.

In various embodiments, the lordosis angle between the upper and lower endplates is adjusted in discrete steps. The gauge further comprises an indicator configured to correspond with each of the discrete steps, which allows the user to adjust the angles between the upper and lower endplates more precisely.

In various embodiments, the instrument further comprises a locking mechanism on the proximal handle. The locking mechanism is configured to prevent the shaft of the adjustment device from coupling to the proximal handle so that the user does not accidently move the endplates relative to each other at the wrong time during the procedure (e.g., during insertion of the implant between the vertebral bodies). In an exemplary embodiment, the locking mechanism comprises a rotatable knob or locking bar on the proximal handle of the instrument.

In various embodiments, the instrument further comprises a third mating feature on the distal end portion for engaging a second translation member within the medical device, and a second adjustment device for moving the second translation member to adjust a distance between the upper and lower endplates of the medical device. The second translation member is configured to adjust the overall height of the medical device by moving the upper and lower endplates towards and away from each other.

In various embodiment, the instrument further comprises a second actuator rod extending through the shaft coupling the second adjustment device with the third mating feature. The second adjustment device comprises a rotatable handle and a shaft. The shaft is configured for insertion into a second proximal opening on the handle of the second adjustment device such that rotation of the shaft of the second adjustment device causes longitudinal translation of the second actuator rod. In some embodiments, the rod is translated proximally to increase this distance and in other embodiments, the rod is translated distally to increase this distance. Translation of the rod, in turn, adjusts a height of the upper and lower endplates of the medical device.

In various embodiments, the instrument further comprises a second gauge on the proximal handle configured to indicate the distance between the distal and proximal ends of the upper and lower endplates. The second gauge comprises a needle and is coupled to the shaft of the second adjustment device such that rotation of said shaft causes rotation of the second needle. In an exemplary embodiment, the distance between the distal and proximal ends of the upper and lower endplates is adjusted in discrete steps and the second gauge further comprises an indicator configured to correspond to each of the discrete steps.

In various embodiments, the first mating feature comprises first and second gripping arms configured to engage and securely attach to a proximal portion of a spinal fusion device. The instrument further comprises an interface on the proximal handle coupled to the gripping arms, which is configured to move the gripping arms laterally towards and away from each other to couple the instrument to the spinal implant.

In still another aspect, a system is provided that comprises a medical device configured for insertion between adjacent vertebral bodies in a patient The medical device may, for example, comprise a temporary trial device or a permanent spinal implant. This device can also comprise upper and lower endplates and a translation member movably disposed between the upper and lower endplates. The system further comprises an instrument having an elongate shaft having a proximal handle and a distal end portion. The instrument comprises a first mating feature on the distal end portion for engaging the translation member and an adjustment device for moving the translation member to adjust an angle between upper and lower endplates of the medical device.

In various embodiments, the translation members comprise one or more angled surfaces or wedges designed to cooperate with angled surfaces or ramps on the endplates to adjust the relative distances between the endplates. In certain embodiments, the translation members each include a locking member that is rotatably coupled to the translation member such that the locking member may be rotated about the longitudinal axis relative to translation member. The locking members include a central bore for receiving an actuator shaft of the instrument that causes longitudinal movement of the translation member. The central bores may include one or more mating features configured to receive, and couple to, one or more mating features of the actuator shaft of the instrument.

In various embodiments, the medical devices and/or spinal implants may include one or more mechanisms for providing discrete steps as the translation members are moved related to the endplates. These steps correlate with discrete angle and/or height adjustments of the endplates. The system may cooperate with gauges on the instrument to provide the user with visual indicators of these discrete angle and/or height adjustments.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure.

FIG. 1 is an exploded view of an exemplary surgical instrument for use with the devices and implants described herein;

FIG. 2A illustrates various components of a surgical instrument system including the instrument of FIG. 1;

FIG. 2B is an enlarged view of a distal end portion of the instrument of FIG. 1;

FIG. 2C is a side sectional view of the distal end portion;

FIG. 3 is an exploded view of certain components of the distal portion of the surgical instrument of FIG. 1;

FIG. 4 is an exploded view of another embodiment of a surgical instrument for use with the spinal devices described herein;

FIG. 5A is a top view of the surgical instrument of FIG. 4 coupled to a spinal implant;

FIG. 5B is an exploded view of certain components of the distal portion of the surgical instrument of FIG. 4;

FIG. 5C is an enlarged view of the distal end of the instrument of FIG. 4;

FIG. 5D illustrates an internal shaft and first and second actuator rods of the instrument of FIG. 4;

FIG. 6 illustrates various components of an exemplary surgical instrument system including the instrument of FIG. 4;

FIG. 7 is a side sectional view of a distal portion of the instrument of FIG. 1 and a perspective view of a lordosis adjustment device;

FIG. 8 is an enlarged side sectional view of the distal end portion of the instrument of FIG. 1;

FIGS. 9A-9C are enlarged views of lordosis and height gauges of the instrument of FIG. 4;

FIG. 10A is a perspective rear view of the instrument of FIG. 4;

FIG. 10B is an enlarged view of the proximal end of the instrument of FIG. 4;

FIG. 11 is a side sectional view of a cage locking screw;

FIGS. 12A and 12B illustrate the surgical instrument of FIG. 4 coupled to a trial device;

FIGS. 13A-13C illustrate the surgical instrument of FIG. 4 adjusting a height of the trial device;

FIG. 14 is a partially transparent side view of a representative trial device;

FIGS. 15A-15C illustrate the surgical instrument of FIG. 4 expanding the height of the trial device and adjusting a lordosis angle of the trial device;

FIG. 16 illustrates the surgical instrument of FIG. 4 with an open gripper interface for coupling to a trial device;

FIG. 17A illustrates the instrument of FIG. 4 coupled to a spinal implant;

FIG. 17B is an enlarged view of a locking bar of the instrument;

FIGS. 18A and 18B illustrate tightening of an implant fixation screw;

FIG. 19 illustrates insertion of the implant into a target site between two vertebral bodies;

FIGS. 20A-20D illustrate expansion of the height of the implant with the surgical instrument;

FIGS. 21A-21C illustrate expansion of the anterior height (lordosis) of the implant with the surgical instrument;

FIGS. 22A and 22B illustrate rotation of the locking bar after the implant's height and angle have been adjusted;

FIG. 23 illustrate opening of a bayonet to remove the expansion unit;

FIG. 24 illustrates removal of the expansion unit;

FIGS. 25A and 25B illustrate an inserter tube for backfilling the implant with graft material;

FIG. 26 illustrates the step of backfilling the implant with graft material;

FIG. 27 illustrates the step of advancing the graft material into the implant;

FIG. 28 is a perspective view of another embodiment of a surgical instrument for use with the spinal devices described herein;

FIG. 29 is an enlarged view of a proximal portion of the surgical instrument of FIG. 28;

FIG. 30 is an enlarged view of another portion of the surgical instrument of FIG. 28;

FIG. 31A is a perspective view of yet another embodiment of a surgical instrument for use with the spinal devices described herein;

FIG. 31B is an enlarged view of a proximal portion of the surgical instrument of FIG. 31A;

FIG. 32A is a perspective view of still another embodiment of a surgical instrument for use with the spinal devices described herein;

FIG. 32B is an enlarged view of a proximal portion of the surgical instrument of FIG. 32A;

FIG. 33A is a perspective view of even still another embodiment of a surgical instrument for use with the spinal devices described herein;

FIG. 33B is an enlarged view of a proximal portion of the surgical instrument of FIG. 33A;

FIG. 34A is a perspective view of even further still another embodiment of a surgical instrument for use with the spinal devices described herein; and

FIG. 34B is an enlarged view of a proximal portion of the surgical instrument of FIG. 34A.

### DETAILED DESCRIPTION OF THE EMBODIMENTS

This description and the accompanying drawings illustrate exemplary embodiments and should not be taken as limiting, with the claims defining the scope of the present disclosure, including equivalents. Various mechanical, compositional, structural, and operational changes may be made without departing from the scope of this description and the claims, including equivalents. In some instances, well-known structures and techniques have not been shown or described in detail so as not to obscure the disclosure. Like numbers in two or more figures represent the same or similar elements. Furthermore, elements and their associated aspects that are described in detail with reference to one embodiment may, whenever practical, be included in other embodiments in which they are not specifically shown or described. For example, if an element is described in detail with reference to one embodiment and is not described with reference to a second embodiment, the element may nevertheless be claimed as included in the second embodiment. Moreover, the depictions herein are for illustrative purposes only and do not necessarily reflect the actual shape, size, or dimensions of the system or illustrated components.

It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

The surgical instruments described herein may be used to assess and/or measure the dimensions of a target space between vertebral bodies and/or to deploy a variety of different temporary trial devices and/or permanent spinal implants, including those described in the patent applications referred to below and incorporated herein by reference. What is meant by “temporary” is that the device or implant being inserted is not intended to be left inside the intervertebral space at the conclusion of the surgical procedure, whereas what is meant by “permanent” is that the device or implant is intended to be left within the intervertebral space at the conclusion of the surgical procedure. The trial and spinal implants may be configured for placement between two vertebral bodies and may be particularly useful for placement from a posterior approach outside of the facet joint (transforaminal lumbar interbody fusion or TLIF), an anterior lumbar interbody fusion (ALIF), a posterior lumbar interbody fusion (PLIF) and/or a lateral lumbar interbody fusion (LLIF).

The implants will generally include upper and lower endplates and one or more internal translation members or wedges for adjusting the height of the implant and/or the anterior height or lordosis of the implant. In some embodiments, the anterior height or lordosis angle of the implants may be adjusted by moving the distal ends of the upper and lower endplates towards and away from each other. In other embodiments, the anterior height or lordosis angle of the implants may be adjusted by moving the lateral sides of the upper and lower endplates away from each other. The height of the implants is generally adjusted by moving the entire upper and lower endplates towards and away from each other.

In some embodiments, the translation members within the implants are movable in the longitudinal direction within internal channels or bores between the endplates. The translation members have one or more angled surfaces or wedges designed to cooperate with angled surfaces or ramps on the endplates to adjust the relative distances between the endplates. In certain embodiments, the translation members each include a locking member that is rotatably coupled to the translation member such that the locking member may be rotated about the longitudinal axis relative to translation member. The locking members include a central bore for receiving an actuator shaft of a surgical instrument (described in detail below) that causes longitudinal movement of the translation member. The central bores may include one or more mating features configured to receive, and couple to, one or more mating features of the actuator shaft of the instrument.

In certain embodiments, the implants may include one or more mechanisms for providing discrete steps as the translation members are moved related to the endplates. These steps correlate with discrete angle and/or height adjustments of the endplates. The system may cooperate with gauges on the instrument (discussed below) to provide the user with visual indicators of these discrete angle and/or height adjustments. A more complete description of such spinal implants can be found in commonly assigned, co-pending U.S. application Ser. No. 17/865,755 and International Patent Application Nos. PCT/EP2022/069886 and PCT/EP2024/051180, the complete disclosures of which are incorporated herein by reference for all purposes.

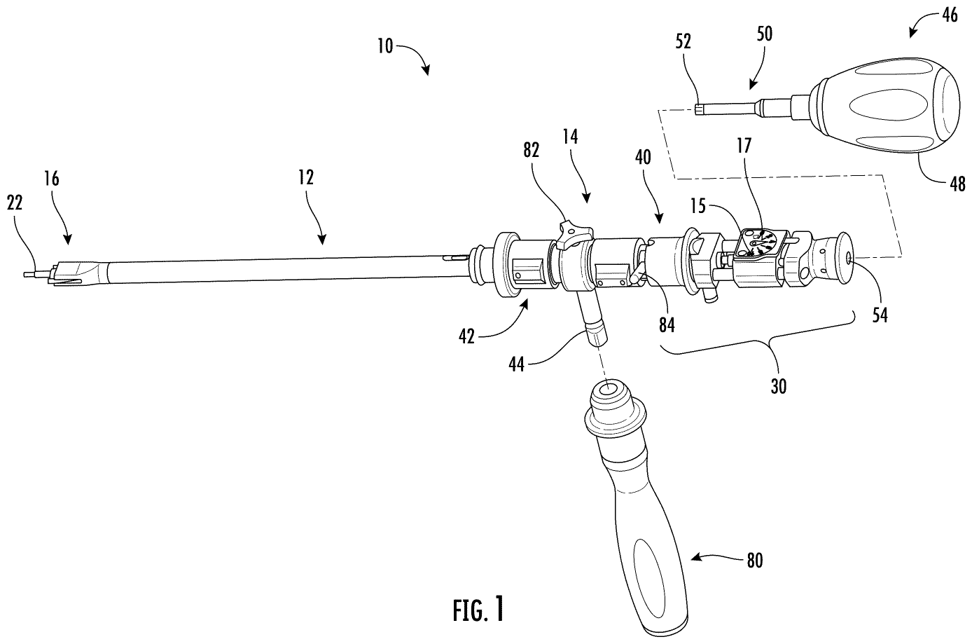

Referring now to FIGS. 1-3, a deployment instrument 10 for use with spinal implants such as the implants and trial devices described above comprises an elongated shaft 12 with a proximal handle 14 and a distal gripping element 16 for removably coupling instrument 10 to a spinal implant (not shown). Instrument 10 may be particularly useful for placement of the implant from a posterior approach outside of the facet joint (transforaminal lumbar interbody fusion or TLIF). Instrument 10 may also be useful for insertion of a trial device through a posterior approach to optimize the size of the spinal implant.

Instrument 10 comprises an actuation unit 30 on the proximal portion of handle 14 for adjusting a lordosis angle or anterior height of the implant (discussed in more detail below). Actuation unit 30 comprises an indicator or gauge 15 on handle 14. Gauge 15 comprises a rotatable needle 17 that provides visual indication of the lordosis angle between the upper and lower endplates of the implant during a trial or implantation procedure.

Instrument 10 further comprises a lordosis adjustment device 46 such as, for example, a torque limiting knob that operates to adjust the lordosis angle of the trial device or implant (discussed below). Device 46 comprises a rotatable handle 48 coupled to a shaft 50 having a mating feature 52 on its distal end. Shaft 50 is configured for insertion into an opening 54 at the proximal end of handle 14. Handle 48 may be rotated by the user relative to shaft 12 to cause an actuator rod 22 to move longitudinally relative to shaft 12. Actuator rod 22, in turn, translates longitudinally through a central bore in the implant to adjust the lordosis angle of the implant. In addition, rotation of handle 48 causes needle 17 on indicator 15 to rotate relative to indicator 15 to display the lordosis angle of the implant. Device 46 is operable to rotate the needle 17 on gauge 15 to match the discrete angle adjustments of the implant such that the user has immediate feedback regarding the lordosis angle of the trial device or the implant.

In some embodiments, actuation unit 30 comprises a first linkage that couples mating feature 52 of device 46 with gauge 15 to rotate needle 17, and a second linkage that coupling gauge 15 with actuator rod 22. In other embodiments, the linkages are reversed such that device 46 is coupled to actuator rod 22 and rod 22 is coupled to gauge 15. In yet another embodiment, actuation unit 30 comprises a single linkage that couples mating feature 52 to both the gauge 15 and the actuator rod 22. A more detailed discussion of this mechanism is discussed below.

In an alternative embodiment, instrument 10 may include a second actuator rod 35 that is coaxial with actuator rod 22 and configured for advancement and retraction through internal bores of an implant (not shown) for cooperating with one or more translation members within the implant to cause longitudinal movement of the translation member(s) to adjust the height of the implant. Actuator rods 22, 35 each include a distal mating feature 23, 37 for coupling to corresponding mating features within the implant. In this embodiment, instrument 10 comprises both a lordosis adjustment device for adjusting a lordosis angle of the implant and a height adjustment device for adjusting the overall height of a trial device or implant. The two adjustment devices may have a similar configuration as devices 140, 150 described below and shown in FIGS. 140, 150. In addition, the instrument may have a similar handle 104 as instrument 100 to actuate the two adjustment devices. A more complete description of the operation of actuator rods 22, 35 and the implant can be found in International Application No. PCT/EP2022/069886, previously incorporated herein by reference.

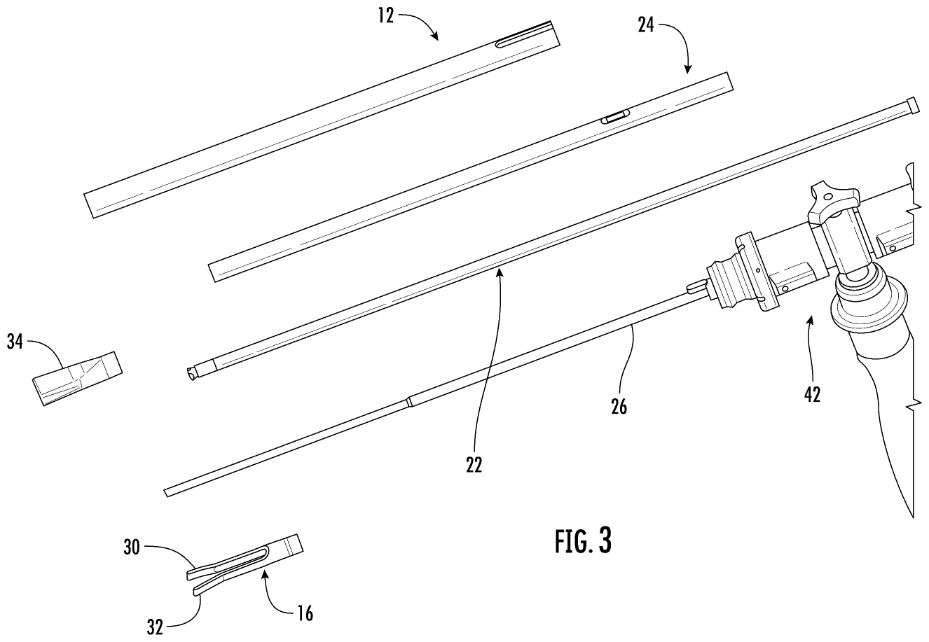

Referring to FIG. 3, elongate shaft 12 includes inner and outer concentric rods 22, 24 surrounding an internal shaft 26. Inner rod 22 is configured for advancement and retraction through an internal bore of implant 20 for cooperating with a translation member within implant 20 to cause longitudinal movement of the translation member to adjust a lordosis angle of implant 20. A more complete description of these elements can be found in International Patent Application No. PCT/EP2024/051180, which is incorporated herein by reference.

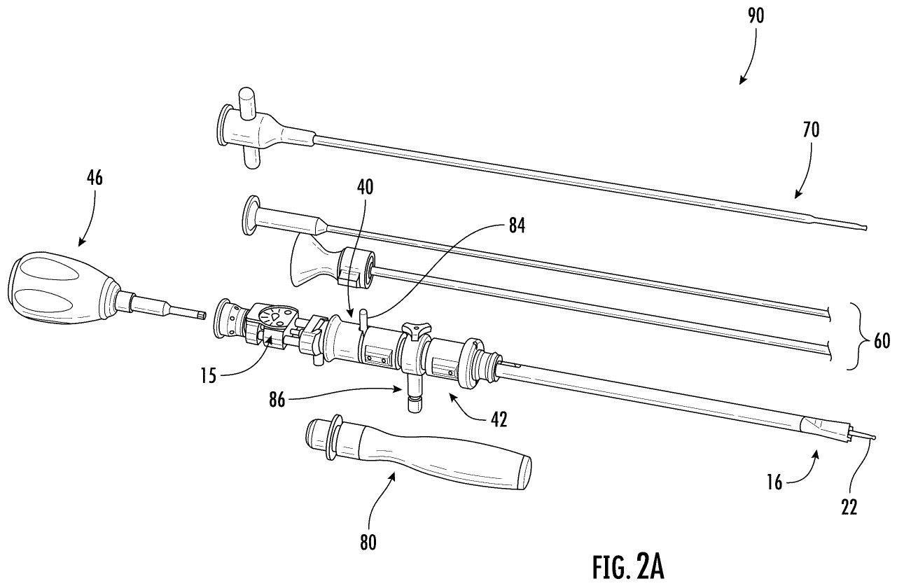

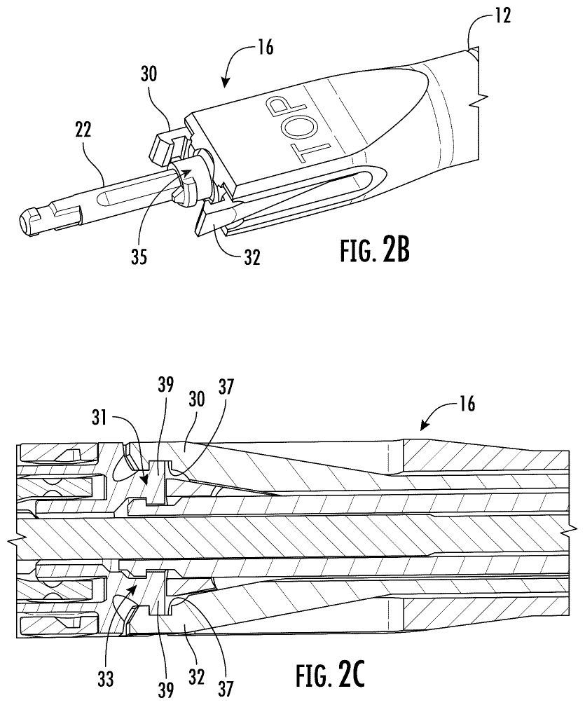

Outer rod 24 is coupled to distal gripping element 16 and may be extended to a proximal end of implant 20 to couple shaft 12 to the implant. In one embodiment, distal gripping element 16 includes first and second gripping arms 30, 32 for coupling to proximal mating features 31, 33 on either side of the upper and/or lower endplates of the implant (see FIGS. 2B and 2C). As shown in FIG. 2C, each of the gripping arms 30, 32 comprises a recessed surface 37 for receiving an enlarged proximal end 39 of each of the mating features 31, 33. Distal gripping arms 30, 32 are coupled to actuator rod 24 to move arms 30, 32 in a substantially lateral direction relative to the longitudinal axis of shaft 12. Arms 30, 32 can be moved together to hold the endplates of the implant and moved apart to release the endplates. In certain embodiments, distal gripping element 16 may further include a sheath 34 that extends over a proximal portion of gripping element 16.

Referring again to FIG. 1, instrument 10 may further include a locking bar 40 on proximal handle 14. Locking bar 40 includes a locking pin 84 extending radially outward from shaft 12 and rotatable relative to shaft 12 from a first position, wherein shaft 50 of lordosis adjustment device 46 may be inserted into opening 54 of handle 14 to adjust the lordosis angle of the implant, to a second position, wherein shaft 50 of lordosis adjustment device 46 cannot be inserted through opening 54 of handle 14. This ensures that the user does not accidently adjust the lordosis angle of the implant at the wrong time during the procedure (e.g., during insertion of the implant between the vertebral bodies).

Instrument 10 further comprises a cage locking screw 42 on proximal handle 14. Cage locking screw 42 is coupled to a proximal end of outer rod 24 and may be used to tighten gripping arms 30, 32 to implant 20. Cage locking screw 42 may comprise any suitable mechanism for tightening gripping arms 30, 32. In an exemplary embodiment, screw 42 comprises a lock-nut with teeth that engage into a rack (not shown) with shaft 102.

As shown in FIG. 2, an instrument assembly 90 includes instrument 10 and may also include a grafting set 60 and a reverse tool 70. The function and operation of these components will be described in more detail below. Instrument 10 may further include a lateral handle 80 coupled to a lateral extension 44 on proximal handle 14 of instrument 10. Lateral handle 80 provides a gripping element for the surgeon to handle and control instrument 10. A rotatable knob 82 is coupled to extension 44 to rotate handle 80 relative to shaft 12. Rotatable knob 82 can be, for example, rotated up to 180 degrees in each direction to rotate handle 80 relative to shaft 12 and facilitate manipulation of instrument 10.

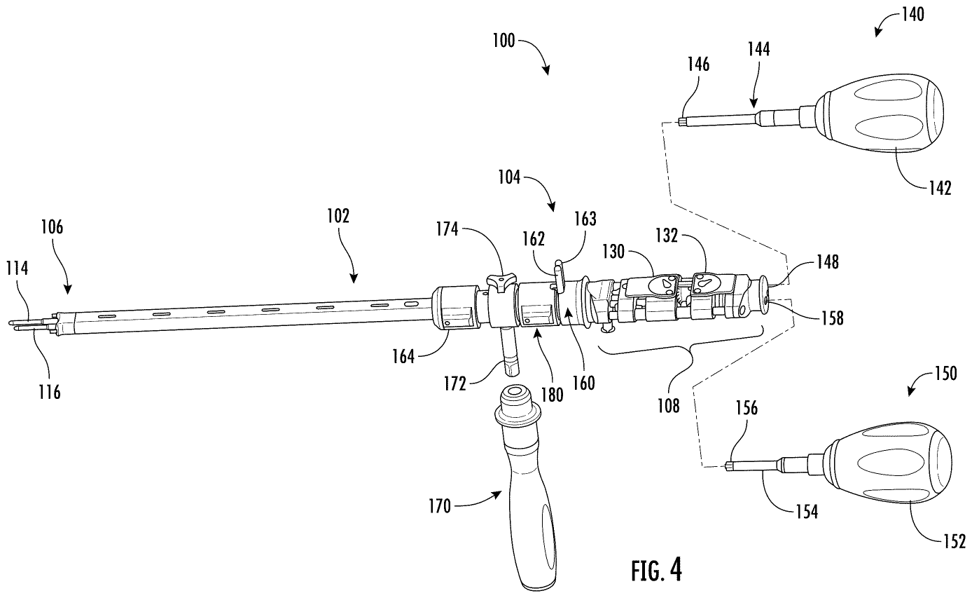

Referring now to FIGS. 4-11, another embodiment of an insertion instrument 100 is provided for use with the implants described above and comprises an elongated shaft 102 with a proximal handle 104 and a distal gripping element 106 for removably coupling instrument 100 to a trial device or spinal implant 120. Instrument 100 may be particularly useful for placement of the trial device or implant 120 from a lateral approach (lateral lumbar interbody fusion (LLIF)). Instrument 100 may also be useful for insertion of a trial device through a lateral approach to optimize the size of the spinal implant.

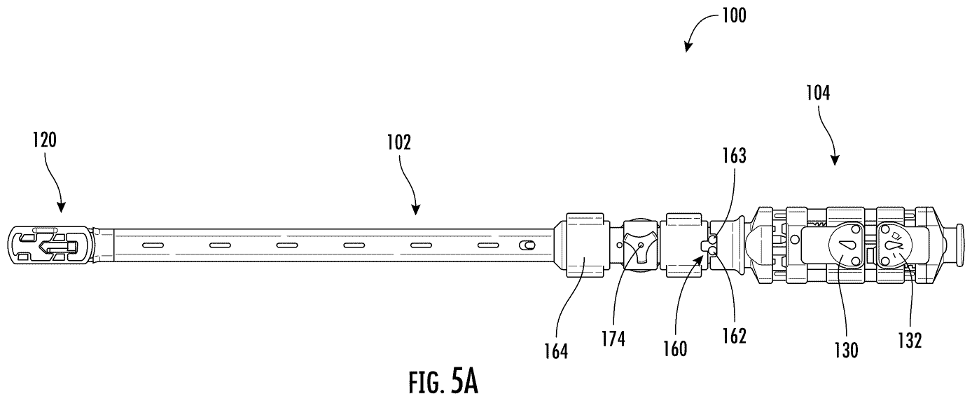

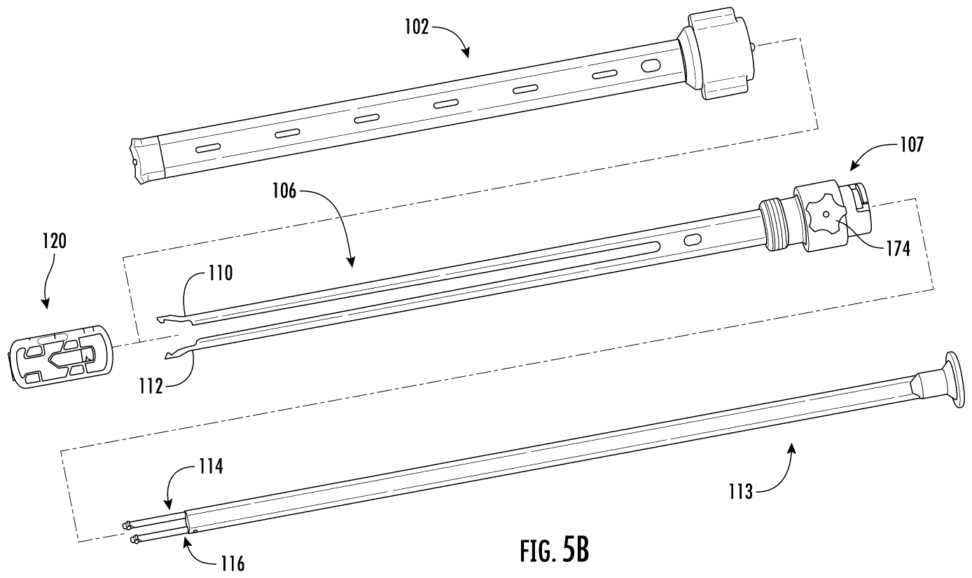

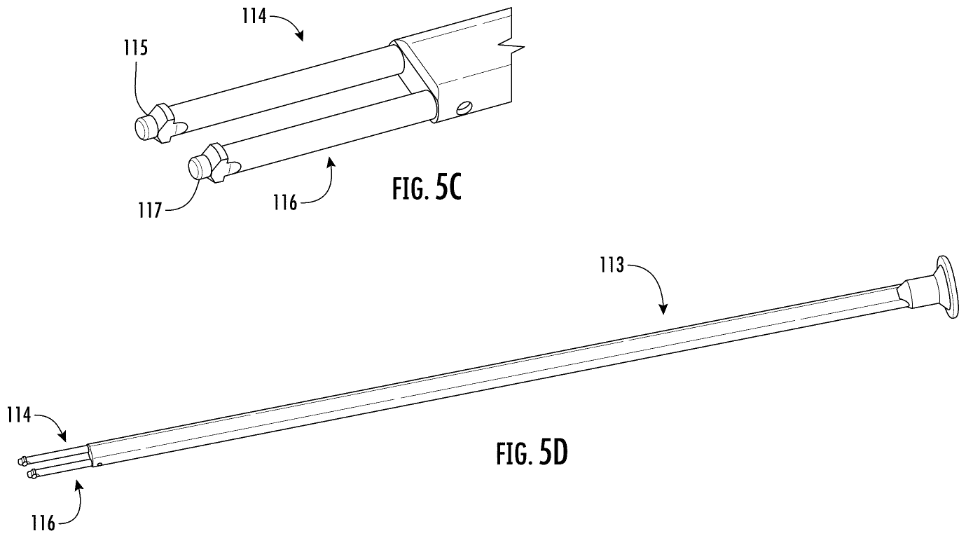

Elongate shaft 102 includes first and second actuator rods 114, 116 configured for advancement and retraction through internal bores of implant 120 for cooperating with one or more translation members within implant 120 to cause longitudinal movement of the translation members to adjust the height and the lordosis angle of implant 120. Actuator rods 114, 116 each include a distal mating feature 115, 117 for coupling to corresponding mating features within implant 120 (see FIG. 5C). A more complete description of these elements can be found in International Patent Application Nos. PCT/EP2024/051180 and PCT/EP2022/069886, incorporated herein by reference.

Instrument 100 further comprises a lordosis adjustment device 140 for adjusting a lordosis angle of implant 120 and a height adjustment device 150 for adjusting the overall height of a trial device or implant 120. Device 140 comprises a rotatable handle 142 coupled to a shaft 144 having a mating feature 146 on its distal end. Shaft 144 is configured for insertion into an opening 148 at the proximal end of handle 104 for adjusting the lordosis angle of the spinal implant (discussed in more detail below). Similarly, height adjustment device 150 comprises a rotatable handle 152 coupled to a shaft 154 having a mating feature 156 on its distal end. Shaft 154 is configured for insertion into an opening 158 at the proximal end of handle 104 for adjusting the height of the spinal implant (discussed in more detail below). In an exemplary embodiment, opening 148 is labeled with a degree of angle or lordosis indicator “L” 190 and opening 158 is labeled with a height indicator “H” 192 to ensure that the operator engages the correct adjustment device with the corresponding actuator rod within shaft 102 (see FIG. 10B). Other embodiments have been considered. For example, these indicators may comprise any suitable alphanumeric symbol, such as an L and an H, numbers (1 or 2), or the like; and/or they may be color-coded to match colors on the shafts of the adjustment devices.

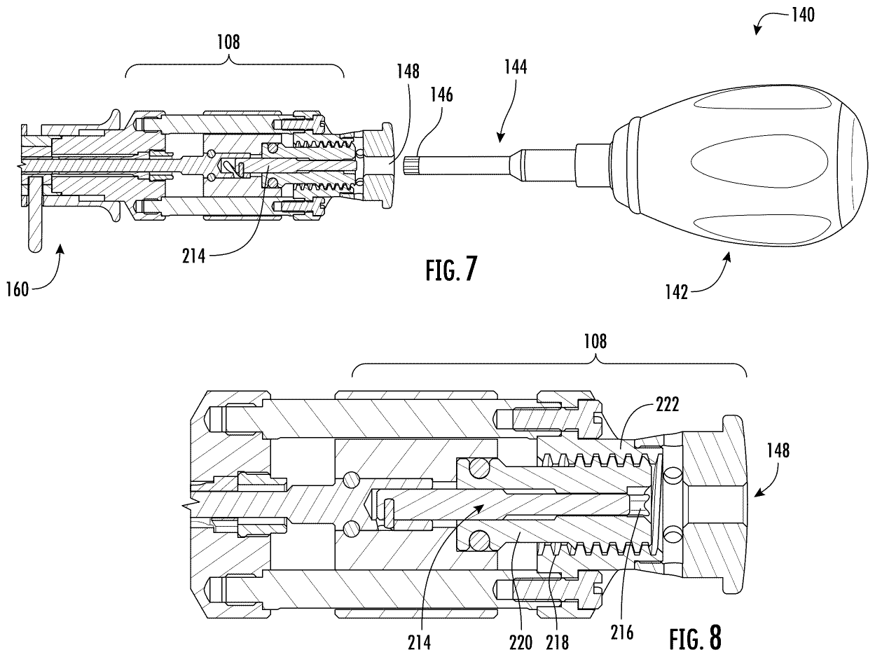

As shown in FIGS. 7 and 8 instrument 100 further comprises an actuation unit 108 in handle 104 for transferring the rotation of devices 140, 150 to longitudinal translation of rods 114, 116. Note that for convenience, FIGS. 7 and 8 only show the section of actuation unit 108 that cooperates with lordosis adjustment device 140, although it should be understood that actuation unit 108 includes a similar construction within proximal handle 104 for cooperating with height adjustment device 150. As shown, actuation unit 108 includes an internal channel 214 that includes a mating feature 216 for engaging and cooperating with mating feature 146 of adjustment device 140. Insertion of mating feature 146 through opening 148 causes this mating feature 146 to engage and secure to mating feature 216. Actuation unit 108 further comprises an outer shaft 220 surrounding channel 214 and comprising external threads 218 that mate with and cooperate with internal threads (not shown) of a hub 222 within handle 104. Rotation of shaft 144 of adjustment device 140 causes rotation of external threads 218 relative to internal threads of hub 222 to longitudinally translate hub 222.

In some embodiments, actuation unit 108 comprises one or more linkages that couple the mating features of the adjustment devices with their corresponding gauges to rotate the needle with the gauges, and one or more linkages that couple the gauges with the corresponding actuator rods. In other embodiments, the linkages are reversed such that the adjustment devices are coupled to the actuator rods and the rods are coupled to the gauge. In yet another embodiment, actuation unit 108 comprises a single linkage that couples each of the mating features to both of the corresponding gauges and actuator rods. In an exemplary embodiment, the linkage comprises a rack and a gear that transfers the linear motion of hub 222 into rotation of the needle on the gauge.

Actuation unit 108 further comprises a first indicator or gauge 130 on handle 104 for providing a user with an indication of a lordosis angle of the implant, and a second indicator or gauge 132 on handle 104 for providing a user an indication of a height of the implant (discussed in more detail below). Device 140, 150 are operable to adjust the indicators on gauge 130, 132, respectively, to match the discrete angle and height adjustments of the implant.

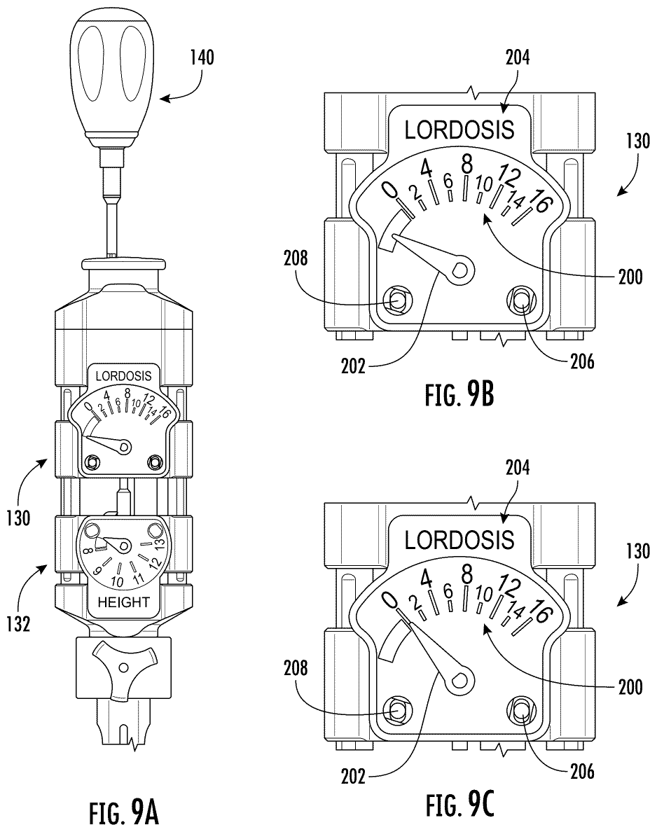

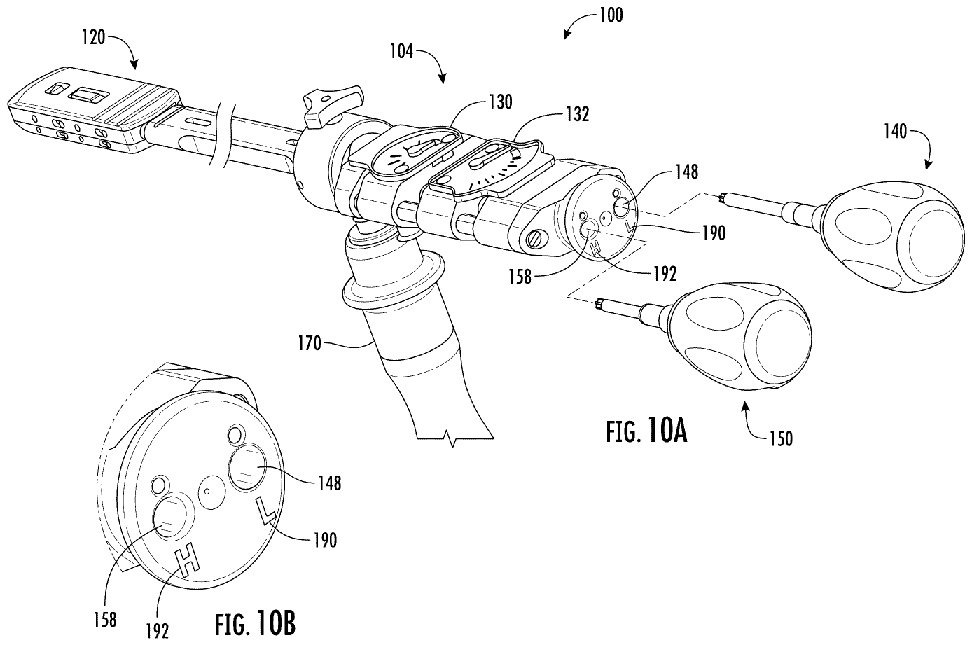

FIGS. 9A-9C show an enlarged view of lordosis and height gauges 130, 132. Each gauge comprises an indicator 200 including numbers that represent discrete steps of height or lordosis adjustment. These numbers correspond with discrete distances moved by the translation members within implant 120. Gauges 130, 132 further comprise a rotatable needle 202 coupled to the mating features 146, 156 of adjustment devices 140, 150 such that rotation of handles 142, 152 causes rotation of the needle 202 to provide the usual with a very clear visual indication of the discrete adjustments made to the implant 120. Gauges 130, 132 also include a visual indicator 204 of each gauge (i.e., height or lordosis). These indicators may comprise any suitable alphanumeric symbol, such as LORDOSIS or HEIGHT, and/or they may be color-coded to match the colors on the shafts of the adjustment devices.

Lordosis and height gauges 130, 132 further comprise locking screws 206, 208 for locking needles 202 at a zero angle position on indicators 200. Thus, the user may adjust needles 202 to ensure that they point to zero prior to adjustment of the height or angle of the implant. After the needles have been “zeroed”, locking screws 206, 208 may be tightened down to ensure that needles 202 do not move unless adjustment devices 140, 150 rotate them during adjustment of the height or lordosis angle of the implant 120.

Referring now to FIG. 5B, actuator rods 114, 116 extend through an internal shaft 113 and are longitudinally translatable relative to shaft 113. Gripping element 106 includes a proximal shaft component 107 with an internal bore for receiving shaft 113 and a rotatable knob 174 on the proximal end of shaft component 107. First and second gripping arms 110, 112 extend distally from proximal shaft component 107, which extends through outer shaft 102. First and second gripping arms 110, 112 are configured to cooperate with proximal mating features on either side of the upper and/or lower endplates of the implant (similar to gripping arms 30, 32 of instrument 10 shown in FIGS. 2B and 2C).

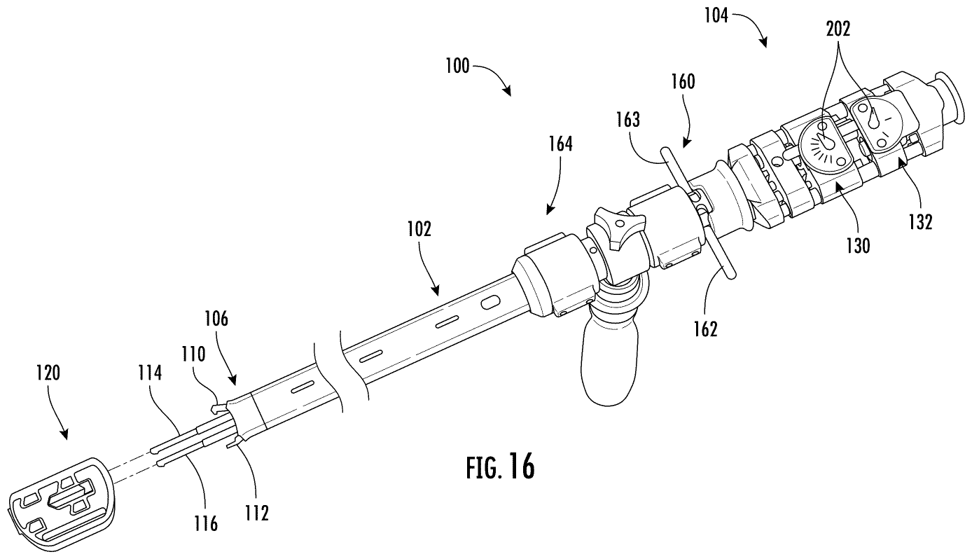

Instrument 100 may further include a locking bar 160 on proximal handle 104. Locking bar 160 includes first and second locking pins 162, 163 extending radially outward from shaft 101 and rotatable relative to shaft 101 between open and closed positions. When locking pin 163 is in the open position (shown in FIG. 16), shaft 146 of lordosis adjustment device 140 may be inserted into opening 148 of handle 104 to adjust the lordosis angle of the implant. When locking pin 163 is in the closed position, shaft 146 of lordosis adjustment device 140 cannot be inserted through opening 148 of handle 104 (or it cannot be mated with the corresponding mating feature in actuator unit 108). Likewise, when locking pin 162 is in the open position, shaft 156 of height adjustment device 150 may be inserted into opening 158 of handle 104 to adjust the height of the implant. When locking pin 162 is in the closed position, shaft 156 of height adjustment device 150 cannot be inserted through opening 158 of handle 104 (or it cannot be mated with the corresponding mating feature in actuator unit 108). This ensures that the user does not accidently adjust the lordosis angle or height of the implant at the wrong time during the procedure (i.e., during insertion of the implant between the vertebral bodies).

In an exemplary embodiment, locking pins 162, 163 function as a key-hole feature. The drivers for rotating the internal mechanisms that have different outer diameters and a different size screw (e.g., torx) drive. In one such embodiment, the drivers have a relatively large diameter and a relatively small torx-drive. In another embodiment, the drivers have a relatively small diameter and relatively large torx-drive. In yet another embodiment, a first driver has a relatively large diameter and a relatively small torx-drive and a second driver has relatively small diameter and relatively large torx-drive.

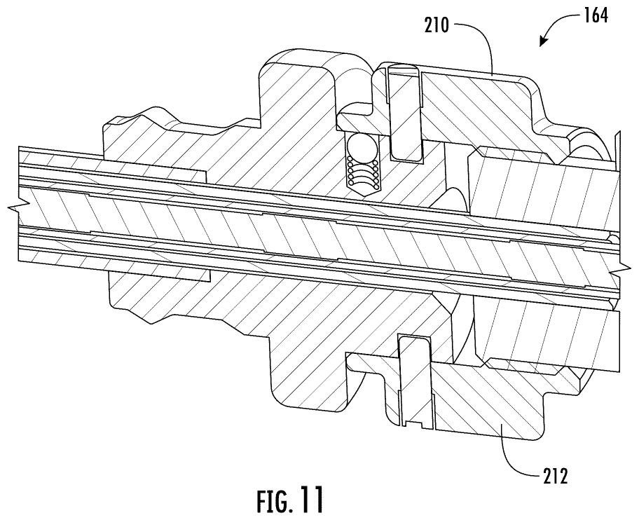

As shown in FIG. 11, instrument 100 further comprises a cage locking screw 164 on proximal handle 104. Cage fixation screw 164 comprises first and second arms 210, 212 extending laterally outward from shaft 102 and configured for gripping by the user to rotate screw 164 relative to shaft 102. Cage fixation screw 164 is coupled to a proximal portion 107 of gripping element 106 and may be used to tighten gripping arms 110, 120 to implant 120. Instrument 100 may further include a coupling mechanism 180, such as a bayonet or the like, for coupling and decoupling proximal handle 104 with shaft 102 (discussed below).

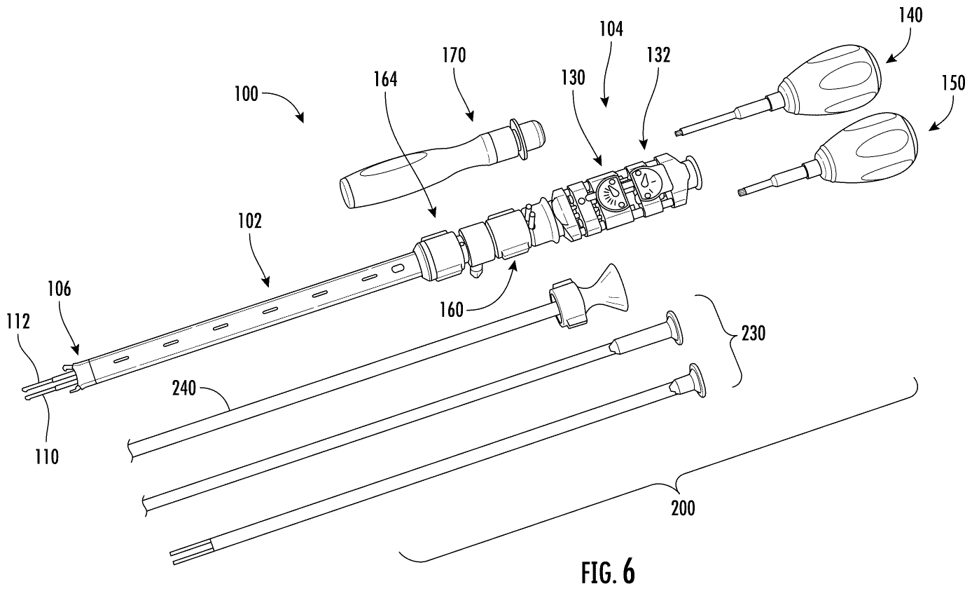

As shown in FIG. 6, an instrument assembly 200 may include instrument 100, a grafting set 230 and a reverse tool 240. The function and operation of these components will be described in more detail below. Instrument 100 may further include a lateral handle 170 coupled to a lateral extension 172 on proximal handle 104 of instrument 100. Lateral handle 170 provides a gripping element for the surgeon to handle and control instrument 100. Rotatable knob 174 allows for rotation of lateral extension and may be rotated at least about 180 degrees in each direction to rotate handle 80 relative to shaft 102 and facilitate manipulation of instrument 100.

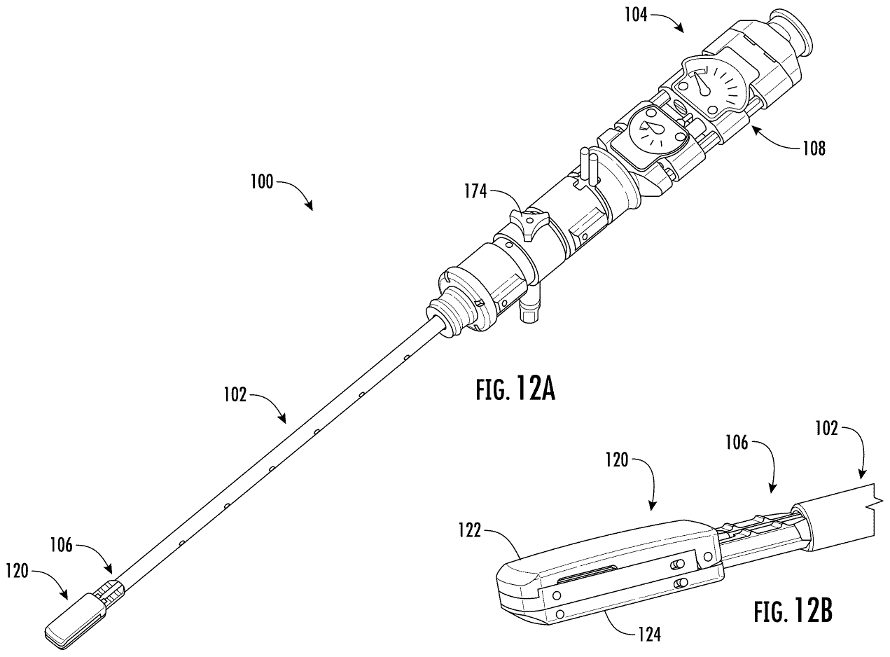

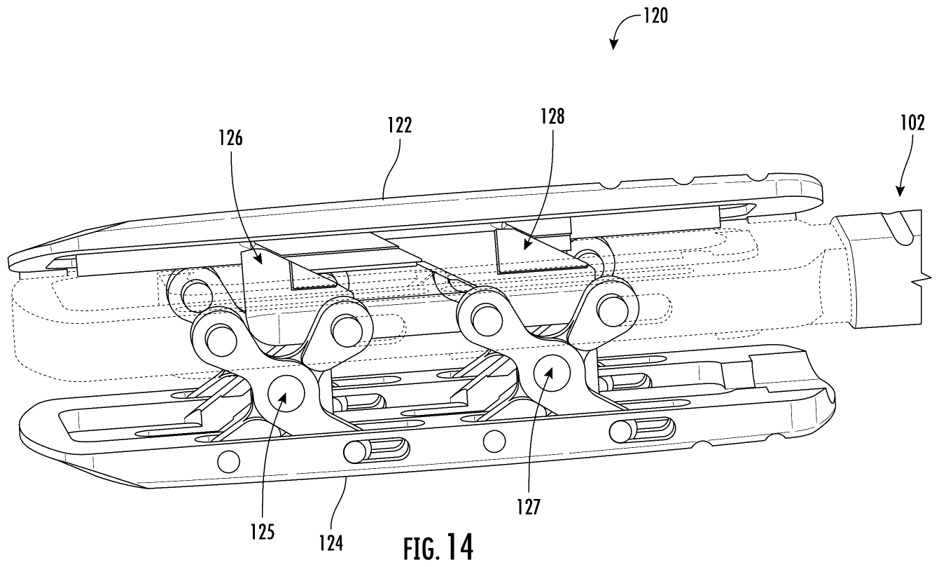

Referring now to FIGS. 12A-15C, instrument 100 may be useful for adjusting a height and/or a lordosis angle of an implant 120 to assess the intervertebral space. FIG. 14 illustrates a representative trial device 120 for use with the instruments described herein. As shown, trial device 120 comprises an upper endplate 122 and a lower endplate 124 coupled together with one or more linkages 125, 127 that allow for movement of upper endplate 122 towards and away from lower endplate 124. It should be understood that linkages 125, 127 represent one embodiment and implant 120 may comprise a variety of different linkages, hinges, or the like for movably coupling upper and lower endplates 122, 124 to each other. Implant 120 further comprises first and second translation members or wedges 126, 128 that cooperate with angled surfaces on upper endplate 122 to adjust height and lordosis. As shown, the distal portion of shaft 102 may advance through a central bore within implant 120 such that actuator rods 114, 116 may mate and cooperate with wedges 126, 128 to translate wedges longitudinally to adjust the height and angle between endplates 122, 124.

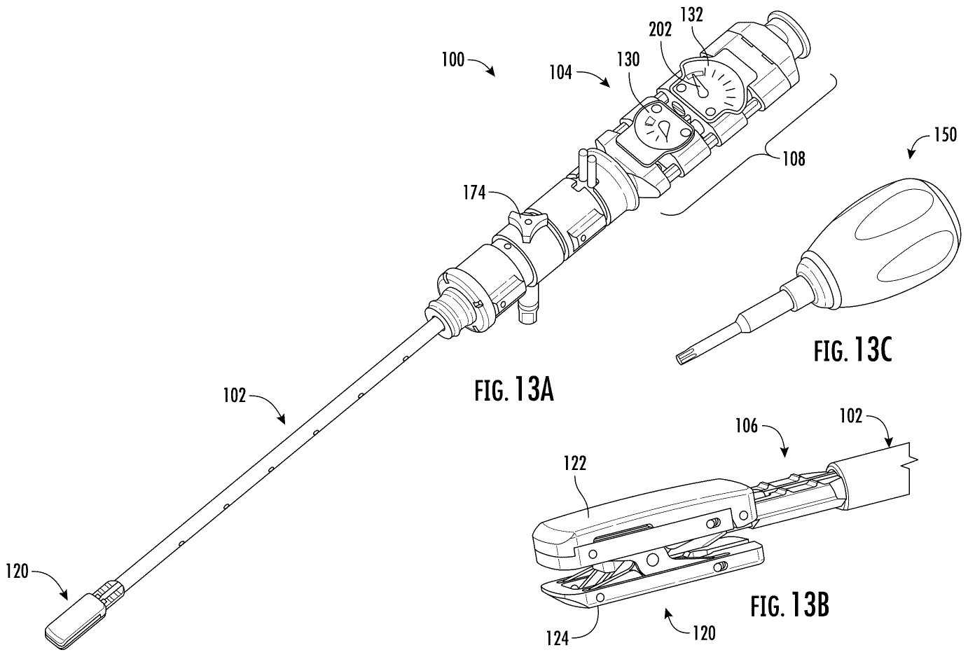

Referring now to FIGS. 13A-13C, adjustment of the height of the implant 120 involves moving first and second endplates 122, 124 of the implant towards and away from each other, which can occur independently or together. For example, in some embodiments, upper endplate 122 is moved relative to lower endplate 124. In other embodiments, lower endplate 124 is moved relative to upper endplate 122. In yet another embodiment, both endplates 122, 124 are moved simultaneously. As discussed previously, height adjustment device 150 is inserted into opening 158 within the proximal end of shaft 102 and rotated. This rotation, in turn, cause longitudinal translation of actuator rod 116 within implant 120 to advance or retract one or more translation members or wedges within the implant. This longitudinal translation of the wedges causes the endplates to move towards and away from each other. In addition, rotation of handle 152 of device 150 causes needle 202 to rotate within gauge 132 such that the height adjustment imparted to the implant is matched by the rotation of needle 202.

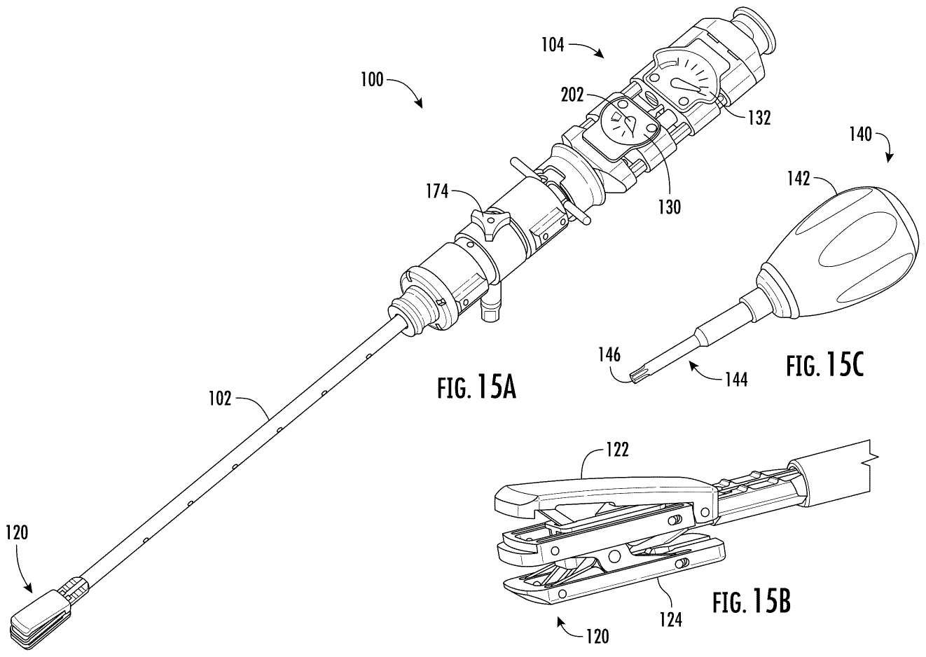

Referring now to FIGS. 15A-15C, in one embodiment, adjustment of the lordosis angle involves moving only the distal portions of the endplates 122, 124 towards and away from each other such that the implant 120 pivots about its proximal portion to adjust the angle between endplates 122, 124 (see FIG. 15B). In other embodiments, adjustment of the lordosis angle may involve rotating the implant laterally such that one of the sides of the upper and lower endplates are moved towards and away from each other. As discussed previously, lordosis adjustment device 140 is inserted into opening 148 within the proximal end of shaft 102 and rotated. This rotation, in turn, cause longitudinal translation of actuator rod 114 within implant 120 to advance or retract one or more translation members or wedges within the implant. This longitudinal translation of the wedges causes the distal ends of the endplates to move towards and away from each other. In addition, rotation of handle 142 of device 140 causes the needle 202 to rotate within gauge 130 such that the angle adjustment imparted to the implant is matched by the rotation of needle 202.

Referring now to FIGS. 16-27, a method for positioning implant 120 within an intervertebral space 300 between adjacent intervertebral bodies 302, 304 of the spine will now be described. As shown in FIG. 16, instrument 100 is first coupled to implant 120 by inserting actuator rods 114, 116 through one or more central bores of implant 120 (discussed above) and attaching gripper arms 110, 112 to mating features on upper and lower endplates 122, 124 of implant 120. At this point in the procedure, locking pins 162, 163 are in the open configuration and needles 202 of gauges 130, 132 are positioned such that they indicate 0 on the gauges 130, 132 (i.e., there has been no adjustment of height or lordosis to the implant). In the event that needles 202 are not pointed to the zero position on gauges 130, 132, the user may move them to that position and then tighten locking screws 206, 208, as discussed previously. Cage fixation screw 164 is also configured in an open position such that gripping arms 110, 112 are open and capable of being positioned around a proximal interface of endplates 122, 124.

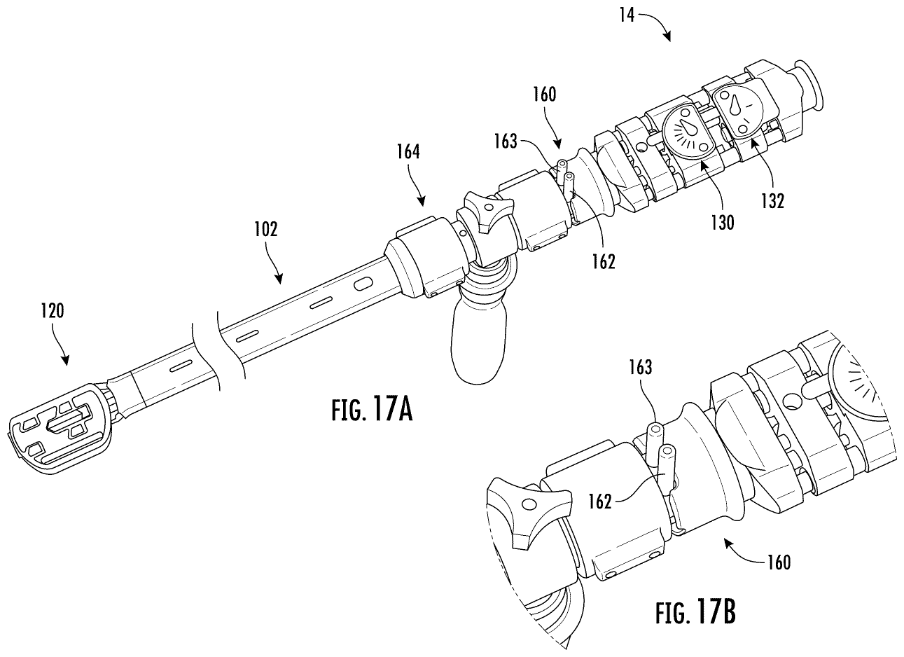

Referring now to FIGS. 17A and 17B, locking pins 162, 163 are then rotated towards the longitudinal axis of shaft 102 until they are generally perpendicular with shaft 102 and extending upwards relative to gauges 130, 132. In this position, the shafts of lordosis and height adjustment devices 140, 150 cannot be inserted into openings 148, 158 of proximal handle such that they engage and cooperate with the internal mechanisms for adjusting height and lordosis. This prevents accidental adjustment of the implant before it has been completely secured to instrument 100 and inserted into the vertebral space 300.

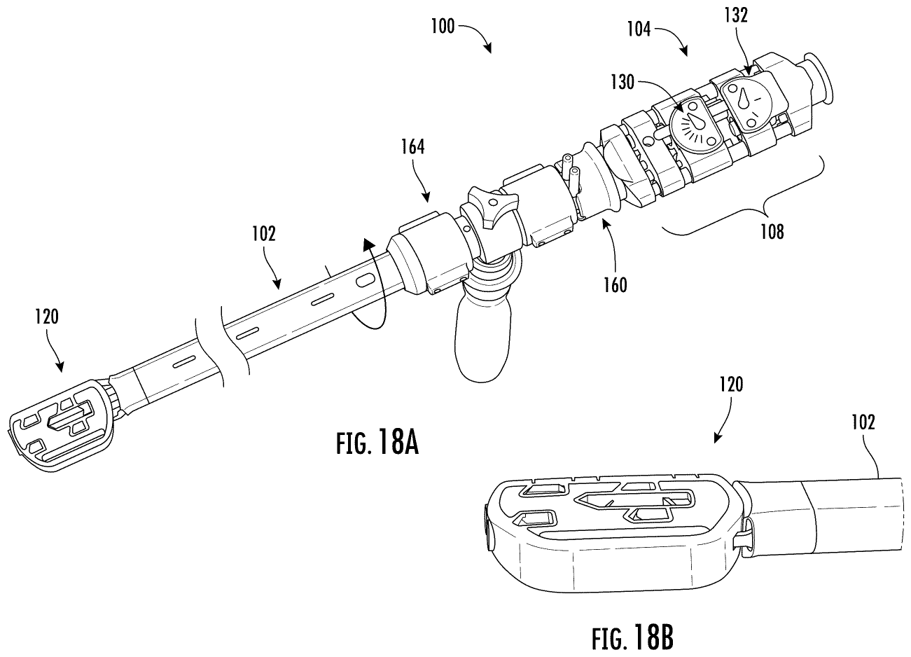

Referring now to FIGS. 18A and 18B, first and second arms 210, 212 of cage locking screw 164 are rotated relative to shaft 102 to tighten gripping arms 110, 112 onto implant 120 Arms 210, 212 engage with mating features on implant 120 to secure instrument 100 to implant 120, as discussed previously in reference to FIGS. 2B and 2C.

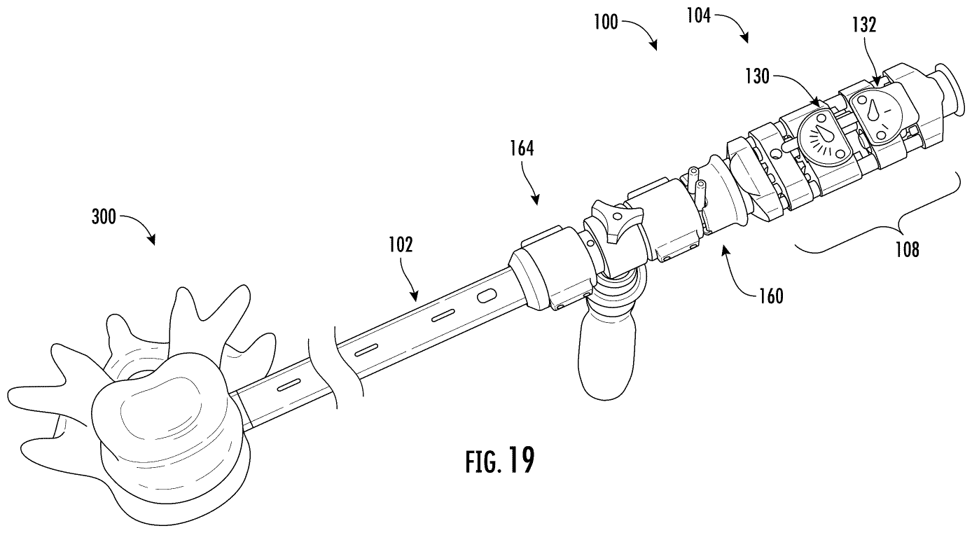

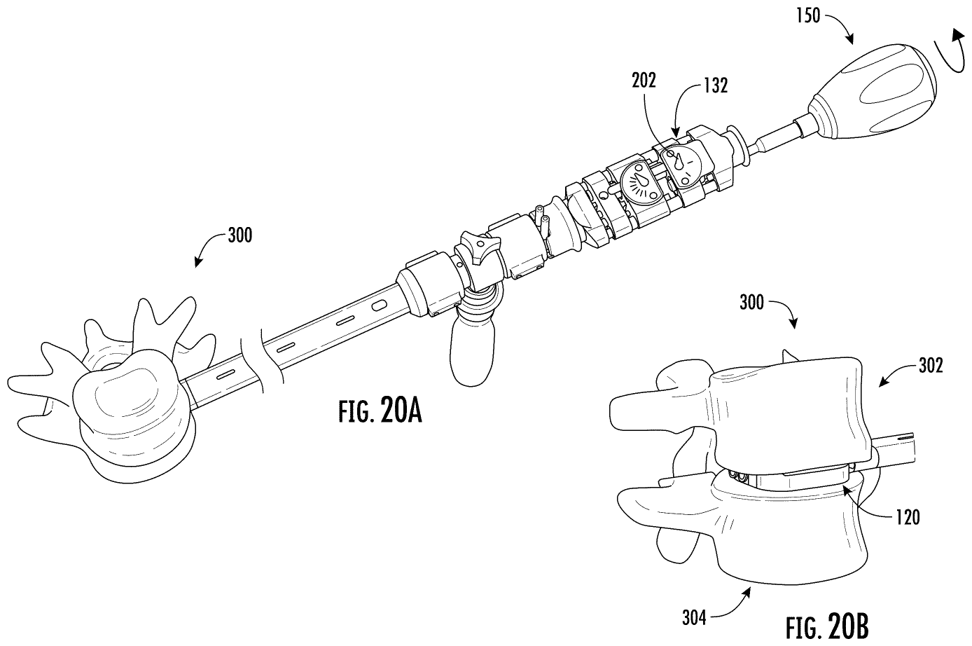

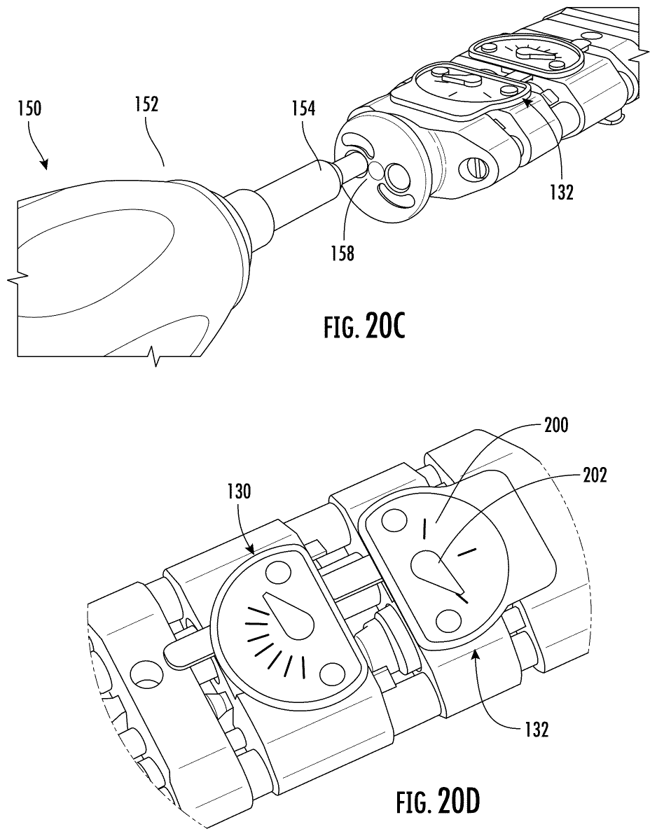

Referring now to FIG. 19, implant 120 is inserted into the vertebral space 300 in a collapsed configuration such that endplates 122, 124 are substantially parallel to each other. As shown in FIGS. 20A-20D, shaft 154 of height adjustment device 150 is then inserted into opening 158 of handle 104 and rotated to adjust the height of implant 120. As discussed above, rotation of handle 152 causes longitudinal translation of actuator rod 116, which causes one or more of the wedges in implant 120 to translate and move endplates 122, 124 apart from each other (see FIG. 20B). In addition, rotation of handle 152 cause the needle 202 on gauge 132 to rotate such that needle 202 indicates the degree or amount of height adjustment imparted to implant 120 (see FIG. 20D).

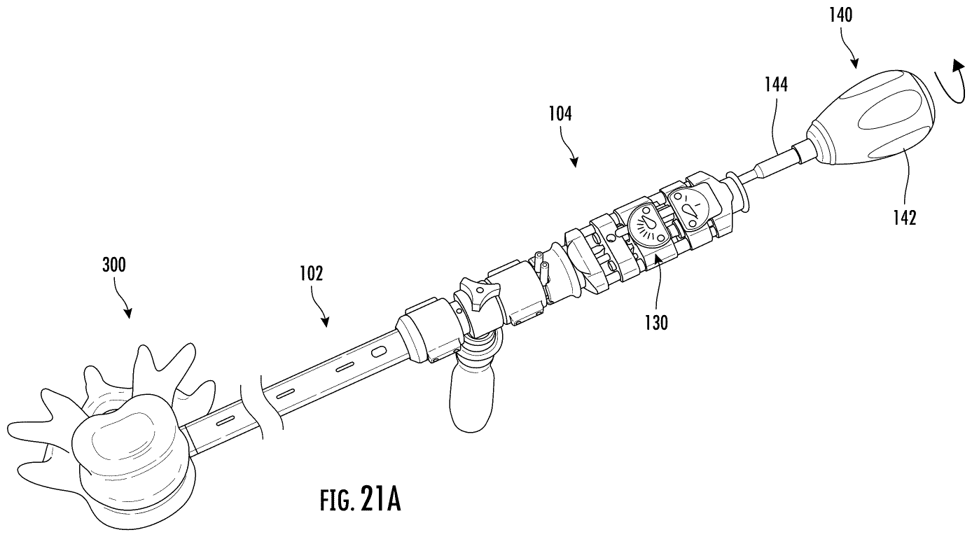

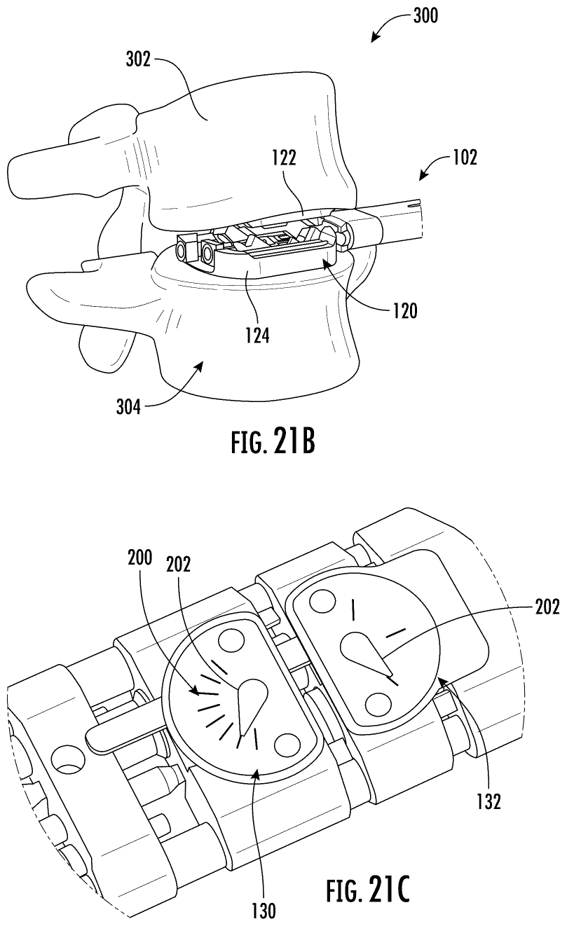

Referring now to FIGS. 21A-21C, shaft 144 of lordosis adjustment device 140 is then inserted into opening 148 of handle 104 and rotated to adjust the lordosis angle or anterior height of implant 120. As discussed above, rotation of handle 142 causes longitudinal translation of actuator rod 114, which causes one or more wedges within implant 120 to translate and move one side of endplates 122, 124 away from each other (see FIG. 21B). In addition, rotation of handle 142 cause the needle 202 on gauge 130 to rotate such that needle 202 indicates the degree or amount of lordosis angle adjustment imparted to implant 120 (see FIG. 21C). In the representative embodiment shown in FIG. 21B, the lateral sides of endplates 122, 124 are moved away from each other. However, it will be recognized that in some embodiments, instrument 100 will move the distal ends of endplates 122, 124 away from each other to adjust the lordosis angle.

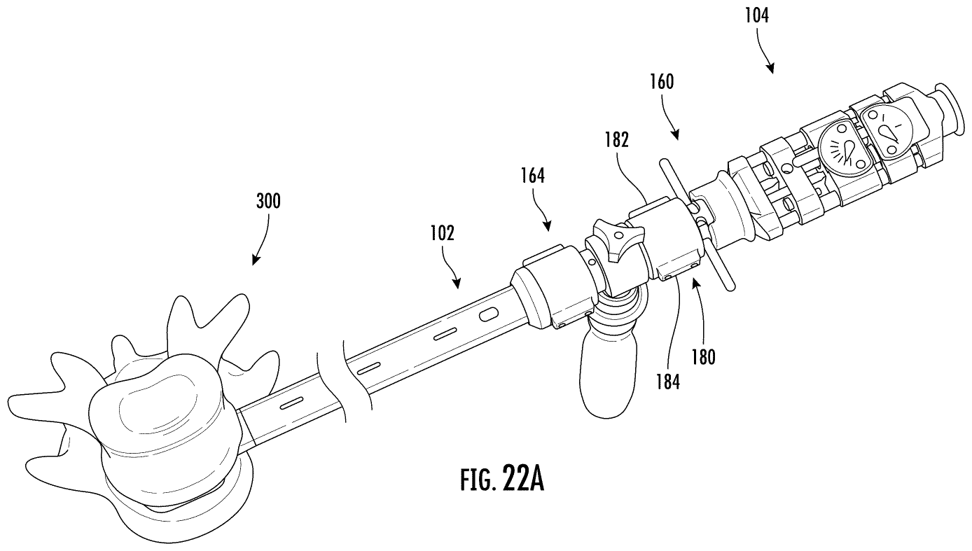

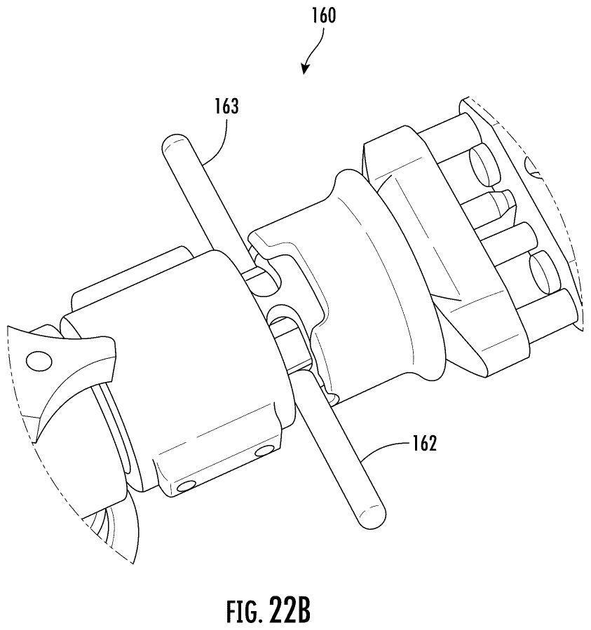

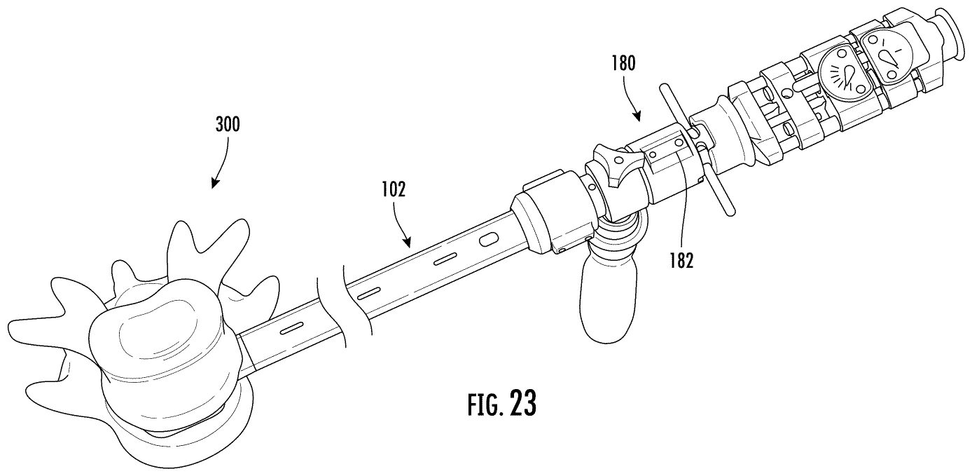

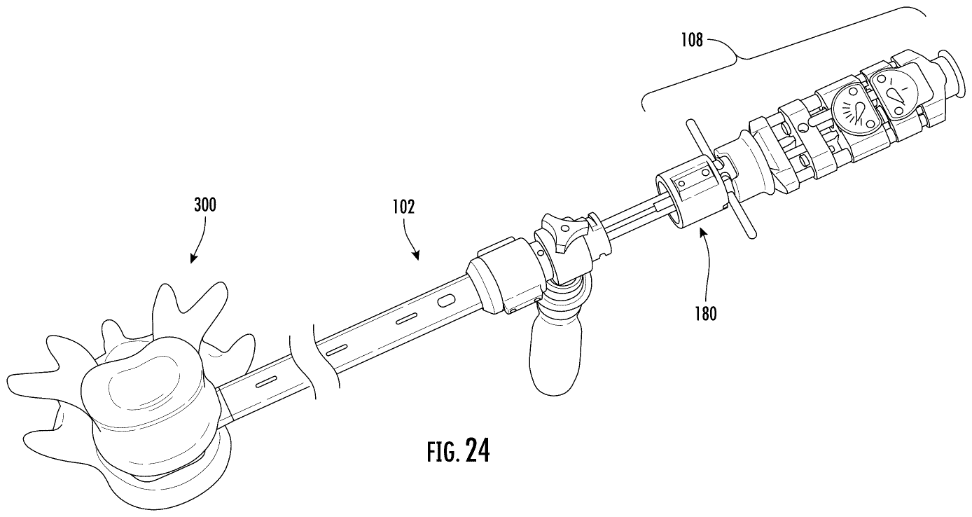

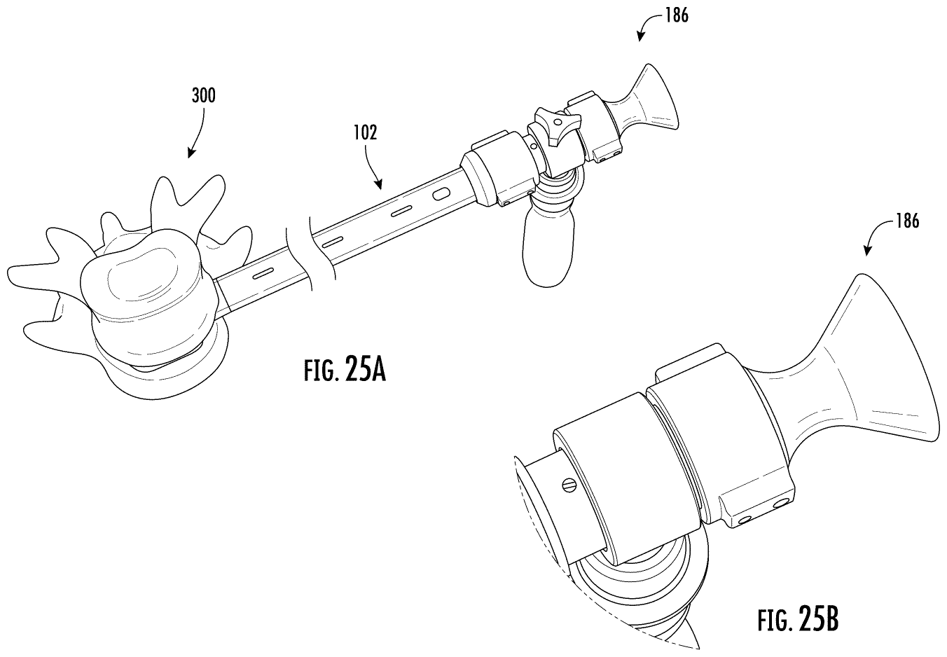

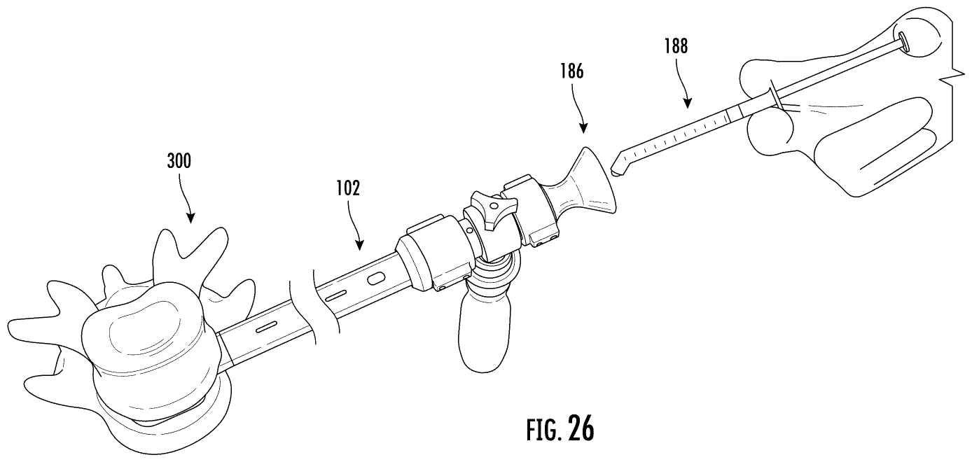

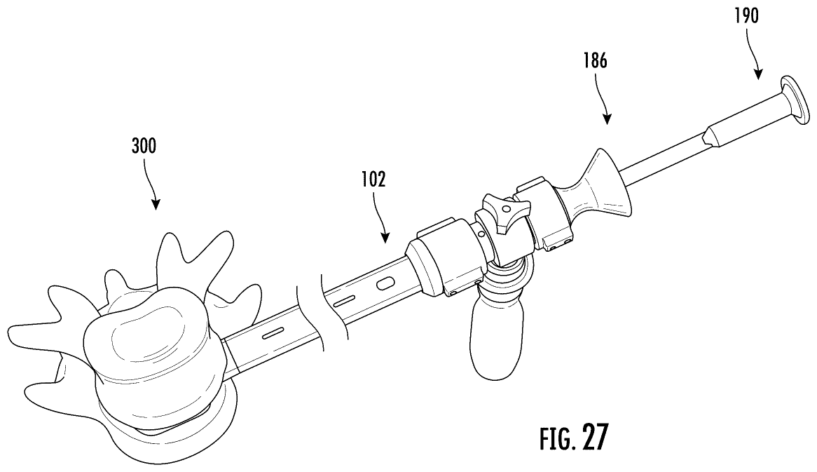

As shown in FIGS. 22A and 22B, adjustment devices 140, 150 are removed from instrument 100 and locking pins 162, 163 are rotated back into their original open position such that the shafts 142, 152 of devices 140, 150 may no longer be inserted into handle 104. Protrusion 182 on bayonet 180 may then be rotated relative to shaft 102 (see FIG. 23) to separate handle 104 from shaft 102 (see FIG. 24). Once handle 104 has been removed, a graft funnel 186 is coupled to the proximal end of shaft 102 (see FIGS. 25A and 25B). Bone graft may then be introduced through shaft 102 and into implant 120 with, for example, a syringe 188 or other suitable method known to those in the art (see FIG. 26). The bone graft may be advanced into implant 120 with a graft pusher 190 or other component (see FIG. 27). Once the physician has completed the lordosis angle adjustment and/or the graft filling step, cage fixation screw 16t4 is loosened to open gripping arms 110, 112 so that the physician may remove the instrument from the patient.

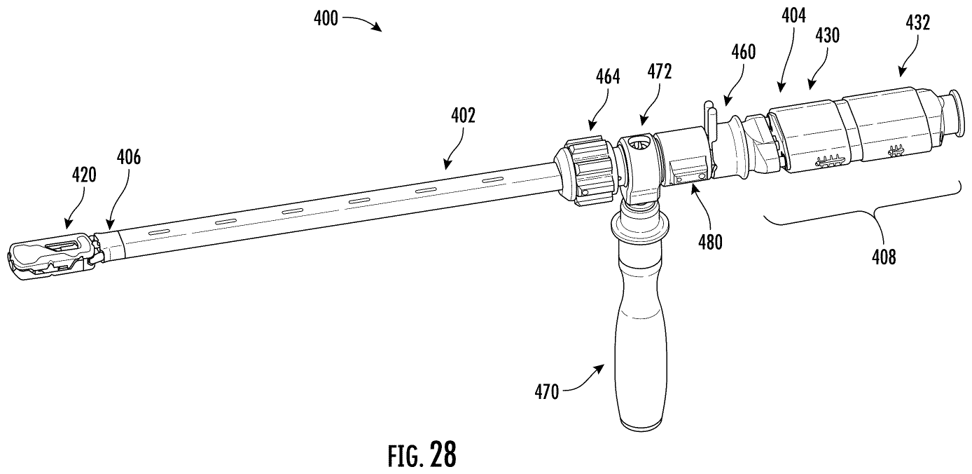

Referring now to FIGS. 28-30, another embodiment of a deployment instrument 400 for use with the implants described above comprises an elongated shaft 402 with a proximal handle 404 and a distal gripping element 406 for removably coupling instrument 400 to a spinal implant 420. Instrument 400 may be particularly useful for placement of an implant 420 from a lateral approach (lateral lumbar interbody fusion (LLIF)). Instrument 400 may also be useful for insertion of a trial device through a lateral approach to optimize the size of the spinal implant.

Similarly to previous embodiments, elongate shaft 402 includes first and second actuator rods (not shown) configured for advancement and retraction through internal bores of implant 420 for cooperating with one or more translation members within implant 420 to cause longitudinal movement of the translation members to adjust the height and the lordosis angle of implant 420. The actuator rods may each include a distal mating feature (not shown) for coupling to corresponding mating features within implant 420 (see FIG. 5C). A more complete description of these elements can be found in International Patent Application Nos. PCT/EP2024/051180 and PCT/EP2022/069886, which are incorporated herein by reference.

Instrument 400 further comprises a lordosis adjustment device for adjusting a lordosis angle of implant 420 and a height adjustment device for adjusting the overall height of implant 420. In some embodiments, these devices are substantially similar in operation and function to devices 140, 150 described above. Instrument 400 further comprises an actuation unit 408 in handle 404 for transferring the rotation of the adjustment devices to longitudinal translation of the actuator rods similar to previous embodiments. In some embodiments, actuation unit 408 comprises one or more linkages that couple the mating features of the adjustment devices with their corresponding gauges to rotate the needle with the gauges, and one or more linkages that couple the gauges with the corresponding actuator rods. In other embodiments, the linkages are reversed such that the adjustment devices are coupled to the actuator rods and the rods are coupled to the gauge. In yet another embodiment, actuation unit 408 comprises a single linkage that couples each of the mating features to both of the corresponding gauges and actuator rods.

Actuation unit 408 further comprises a first indicator or gauge 430 on handle 404 for providing a user with an indication of a lordosis angle of the implant, and a second indicator or gauge 432 on handle 404 for providing a user an indication of a height of the implant (discussed in more detail below). The adjustment devices are operable to adjust the indicators on gauge 430, 432, respectively, to match the discrete angle and height adjustments of the implant.

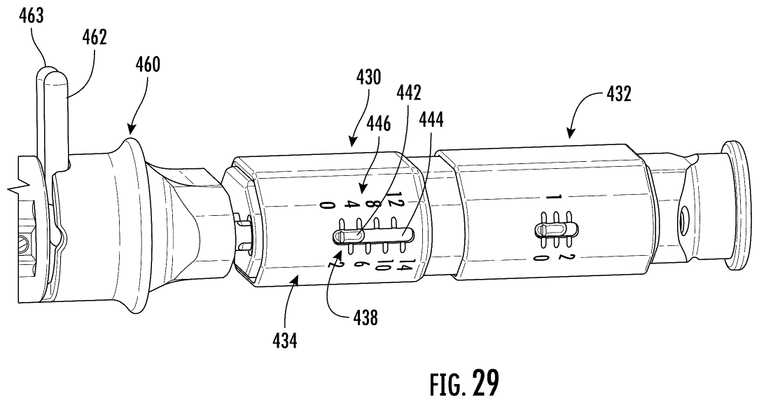

As shown more clearly in FIG. 29, each gauge 430, 432 comprises an annular or circular housing 434 that extends around a portion of proximal handle 404. Housings 434 each include an indicator 438 including numbers 446 that represent discrete steps of height or lordosis adjustment. These numbers correspond with discrete distances moved by the translation members within implant 420. Gauges 430, 432 further comprise a slider 442 coupled to the adjustment devices such that rotation of the handles of these devices causes slide to move longitudinally through a slot 444 in housings 434 to provide the user with a very clear visual indication of the discrete adjustments made to the implant 420. Gauges 430, 432 may also include a visual indicator of each gauge (i.e., height or lordosis). These indicators may comprise any suitable alphanumeric symbol, such as LORDOSIS or HEIGHT, and/or they may be color-coded to match the colors on the shafts of the adjustment devices.

Lordosis and height gauges 430, 432 may further comprise a mechanism (such as locking screws (not shown)) for locking sliders 442 at a zero angle position on indicators 438. Thus, the user may adjust sliders 442 to ensure that they point to zero prior to adjustment of the height or angle of the implant. After the sliders have been “zeroed”, the locking screws may be tightened down to ensure that sliders 442 do not move unless the adjustment devices displace them during adjustment of the height or lordosis angle of the implant 420.

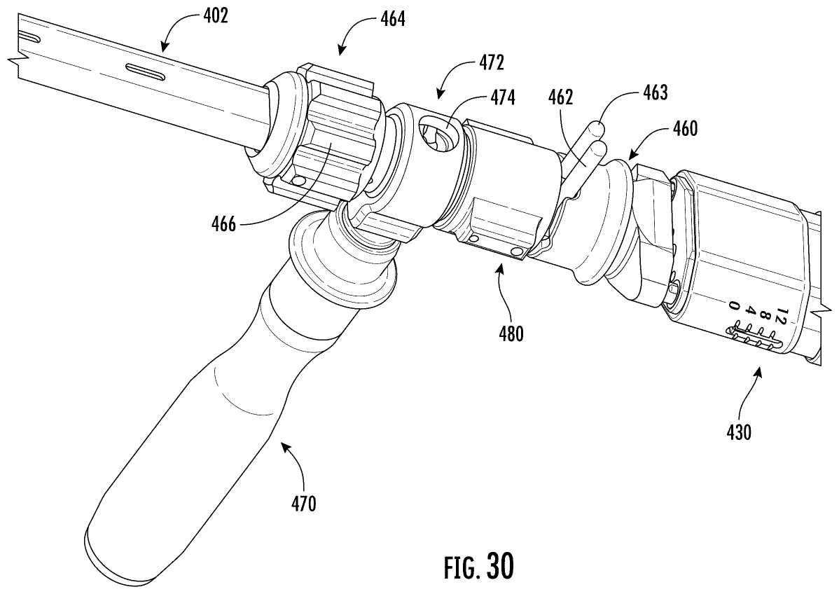

Instrument 400 may further include a locking bar 460 on proximal handle 404. Locking bar 460 includes first and second locking pins 462, 463 extending radially outward from shaft 402 and rotatable relative to shaft 402 between open and closed positions (see FIG. 30). Locking pins 462, 463 operate to control insertion of the adjustment devices with handle 404, similar to locking pins 162, 163 discussed above.

Instrument 400 further comprises a cage locking screw 464 on proximal handle 404. As shown in FIG. 30, cage fixation screw 464 comprises one or more gripping surfaces or protrusions 466 extending laterally outward from shaft 402 and configured for gripping by the user to rotate screw 464 relative to shaft 402. Cage fixation screw 464 is coupled to a proximal portion of gripping element 406 and may be used to tighten the gripping arms of element 406 to implant 420. Instrument 400 may further include a coupling mechanism 480, such as a bayonet or the like, for coupling and decoupling proximal handle 404 with shaft 402. Instrument 400 may further include a lateral handle 470 coupled to a lateral extension 472 on proximal handle 404 of instrument 400. Lateral handle 470 provides a gripping element for the surgeon to handle and control instrument 400. Lateral handle 470 may further include a mechanism to allow handle 470 to be rotated relative to shaft 402. In one embodiment, this mechanism comprises a mating feature 474 that may be engaged with a tool or other device to either rotate handle 470 or remove handle 470 from instrument 400.

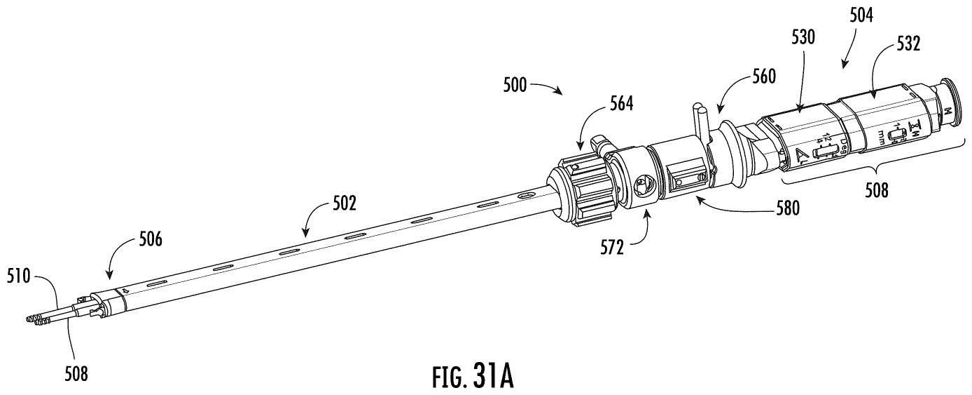

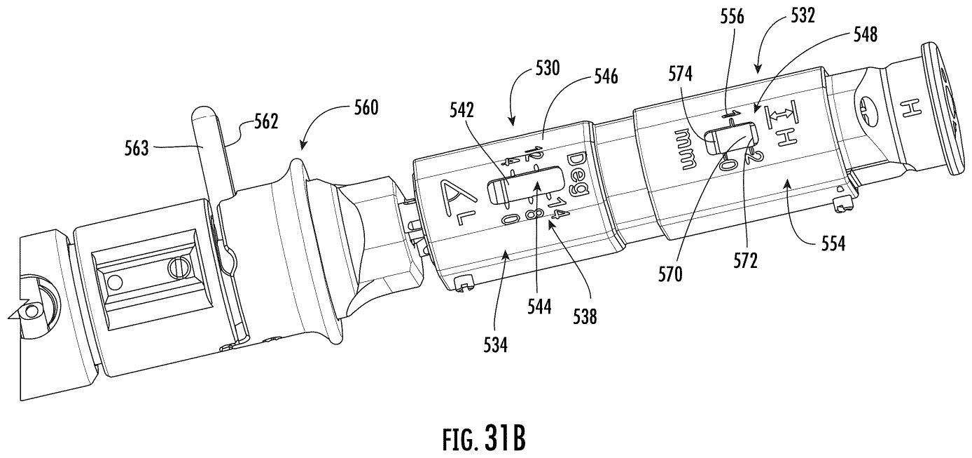

Referring now to FIGS. 31A and 31B, another embodiment of a deployment instrument 500 for use with the implants described above comprises an elongated shaft 502 with a proximal handle 504 and a distal gripping element 506 for removably coupling instrument 500 to a spinal implant (not shown). Instrument 500 may be particularly useful for placement of an implant from a lateral approach (lateral lumbar interbody fusion (LLIF)).

Similarly to previous embodiments, elongate shaft 502 includes first and second actuator rods 508, 510 configured for advancement and retraction through internal bores of the implant for cooperating with one or more translation members within the implant to cause longitudinal movement of the translation members to adjust the height and the lordosis angle of the implant. The actuator rods may each include a distal mating feature (not shown) for coupling to corresponding mating features within the implant (see FIG. 5C). A more complete description of these elements can be found in International Patent Application Nos. PCT/EP2024/051180 and PCT/EP2022/069886, which are incorporated herein by reference.

Instrument 500 further comprises a lordosis adjustment device for adjusting a lordosis angle of the implant and a height adjustment device for adjusting the overall height of the implant. In embodiments, these devices are substantially similar in operation and function to devices 140, 150 described above. Instrument 500 further comprises an actuation unit 508 in handle 504 for transferring the rotation of the adjustment devices to longitudinal translation of 5he actuator rods similar to previous embodiments. In some embodiments, actuation unit 508 comprises one or more linkages that couple the mating features of the adjustment devices with their corresponding gauges to rotate the needle with the gauges, and one or more linkages that couple the gauges with the corresponding actuator rods. In other embodiments, the linkages are reversed such that the adjustment devices are coupled to the actuator rods and the rods are coupled to the gauge. In yet another embodiment, actuation unit 508 comprises a single linkage that couples each of the mating features to both of the corresponding gauges and actuator rods.

Actuation unit 508 further comprises a first indicator or gauge 530 on handle 504 for providing a user with an indication of a lordosis angle of the implant, and a second indicator or gauge 532 on handle 504 for providing a user an indication of a height of the implant (discussed in more detail below). The adjustment devices are operable to adjust the indicators on gauge 530, 532, respectively, to match the discrete angle and height adjustments of the implant.

As shown more clearly in FIG. 31B, each gauge 530, 532 comprises an annular or circular housing 534, 554 that extends around a portion of proximal handle 504. Housings 534, 554 each include an indicator 538, 548 including numbers 546, 556 that represent discrete steps of height or lordosis adjustment. These numbers correspond with discrete distances moved by the translation members within the implant. For example, numbers 546 indicate the angle between the upper and lower endplates of the implant (e.g., from 0 to 14 degrees) and numbers 556 indicate the height or distance between the endplates (e.g., from 0 to 2 mm). Gauges 530, 532 further comprise a slider 542, 574 coupled to the adjustment devices such that rotation of the handles of these devices causes slider to move longitudinally through a slot 544, 570 in housings 534, 554 to provide the user with a very clear visual indication of the discrete adjustments made to the implant. Gauges 530, 532 may also include a visual indicator of each gauge (i.e., height or lordosis). These indicators may comprise any suitable alphanumeric symbol, such as LORDOSIS or HEIGHT, and/or they may be color-coded to match the colors on the shafts of the adjustment devices. In the exemplary embodiment, gauge 530 includes the words Deg and a picture of an angle with the subscript L, for lordosis angle. Similarly, gauge 532 includes the letter H with an arrow indicating that this gauge represents an increase or decrease in height. Guage 532 also includes the letters mm, to indicate that the height numbers 556 are in millimeters.

Lordosis and height gauges 530, 532 may further comprise a mechanism (such as locking screws (not shown)) for locking sliders 542, 574 at a zero angle position on indicators 538, 548. Thus, the user may adjust sliders 542, 574 to ensure that they point to zero prior to adjustment of the height or angle of the implant. After the sliders have been “zeroed”, the locking screws may be tightened down to ensure that sliders 542, 574 do not move unless the adjustment devices displace them during adjustment of the height or lordosis angle of the implant.

Instrument 500 may further include a locking bar 560 on proximal handle 504. Locking bar 560 includes first and second locking pins 562, 563 extending radially outward from shaft 502 and rotatable relative to shaft 502 between open and closed positions (see FIG. 31B). Locking pins 562, 563 operate to control insertion of the adjustment devices with handle 504, similar to locking pins 562, 563 discussed above.

Similar to previous embodiments, instrument 500 further comprises a cage locking screw 564 on proximal handle 504. Cage locking screw 564 is coupled to a proximal portion of gripping element 506 and may be used to tighten the gripping arms of element 506 to the implant. Instrument 500 may further include a coupling mechanism 580, such as a bayonet or the like, for coupling and decoupling proximal handle 504 with shaft 502. Instrument 500 may further include a lateral handle (not shown) coupled to a lateral extension 572 on proximal handle 504 of instrument 500. The lateral handle provides a gripping element for the surgeon to handle and control instrument 500.

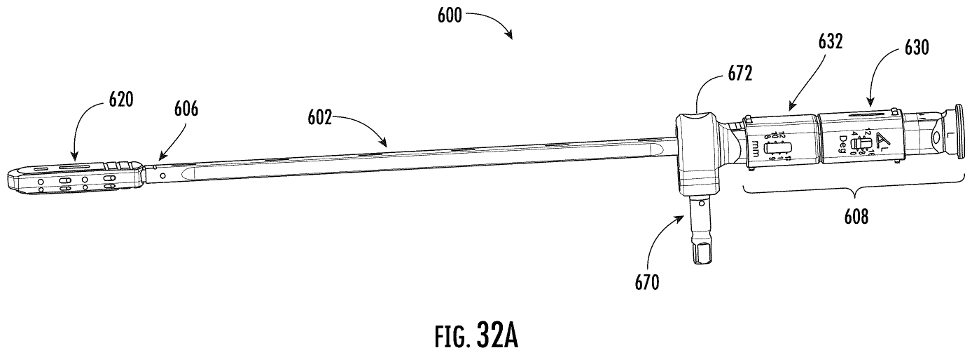

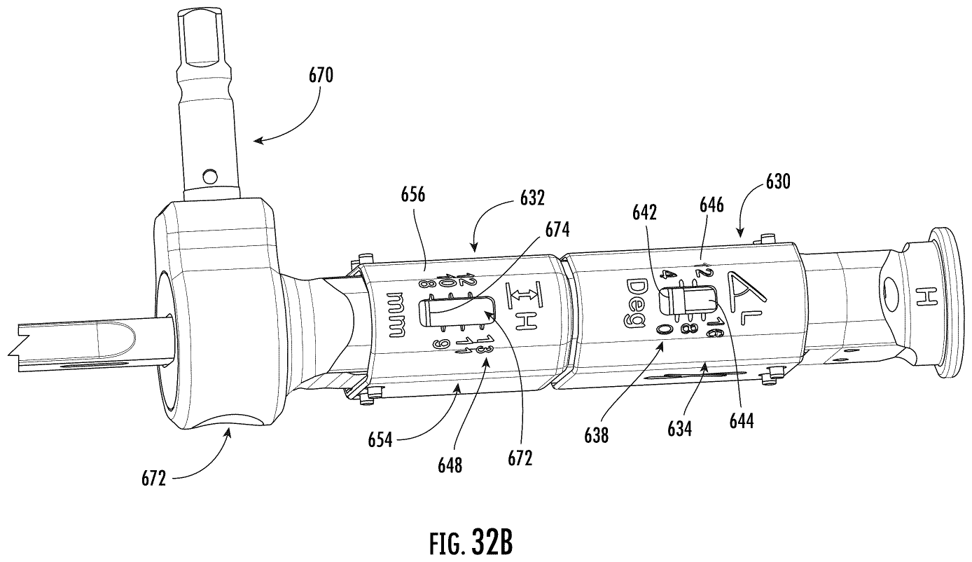

Referring now to FIGS. 32A and 32B, another embodiment of a deployment instrument 600 for use with the implants described above is provided and comprises an elongated shaft 602 with a proximal handle 604 and a distal gripping element 606 for removably coupling instrument 600 to a spinal implant (not shown). Instrument 600 may also be particularly useful for insertion of a trial device 620 through a lateral approach to optimize the size of the spinal implant.

Similarly to previous embodiments, elongate shaft 602 includes first and second actuator rods (not shown) configured for advancement and retraction through internal bores of the implant for cooperating with one or more translation members within the implant to cause longitudinal movement of the translation members to adjust the height and the lordosis angle of the implant. The actuator rods may each include a distal mating feature (not shown) for coupling to corresponding mating features within the implant (see FIG. 5C). A more complete description of these elements can be found in International Patent Application Nos. PCT/EP2024/051180 and PCT/EP2022/069886, which are incorporated herein by reference.

Instrument 600 further comprises a lordosis adjustment device for adjusting a lordosis angle of the implant and a height adjustment device for adjusting the overall height of the implant. In some embodiments, these devices are substantially similar in operation and function to devices 140, 150 described above. Instrument 600 further comprises an actuation unit 608 in handle 604 for transferring the rotation of the adjustment devices to longitudinal translation of the actuator rods similar to previous embodiments. In some embodiments, actuation unit 608 comprises one or more linkages that couple the mating features of the adjustment devices with their corresponding gauges to rotate the needle with the gauges, and one or more linkages that couple the gauges with the corresponding actuator rods. In other embodiments, the linkages are reversed such that the adjustment devices are coupled to the actuator rods and the rods are coupled to the gauge. In yet another embodiment, actuation unit 608 comprises a single linkage that couples each of the mating features to both of the corresponding gauges and actuator rods.

Actuation unit 608 further comprises a first indicator or gauge 630 on handle 604 for providing a user with an indication of a lordosis angle of the implant, and a second indicator or gauge 632 on handle 604 for providing a user an indication of a height of the implant (discussed in more detail below). The adjustment devices are operable to adjust the indicators on gauge 630, 632, respectively, to match the discrete angle and height adjustments of the implant.

As shown more clearly in FIG. 32B, each gauge 630, 632 comprises an annular or circular housing 634, 654 that extends around a portion of proximal handle 604. Housings 634, 654 each include an indicator 638, 648 including numbers 646, 656 that represent discrete steps of height or lordosis adjustment. These numbers correspond with discrete distances moved by the translation members within the implant. For example, numbers 646 indicate the angle between the upper and lower endplates of the implant (e.g., from 0 to 14 degrees) and numbers 656 indicate the height or distance between the endplates (e.g., from 0 to 2 mm). Gauges 630, 632 further comprise a slider 642, 674 coupled to the adjustment devices such that rotation of the handles of these devices causes slider to move longitudinally through a slot 644, 672 in housings 634, 564 to provide the user with a very clear visual indication of the discrete adjustments made to the implant. Gauges 630, 632 may also include a visual indicator of each gauge (i.e., height or lordosis). These indicators may comprise any suitable alphanumeric symbol, such as LORDOSIS or HEIGHT, and/or they may be color-coded to match the colors on the shafts of the adjustment devices. In the exemplary embodiment, gauge 630 includes the words Deg and a picture of an angle with the subscript L, for lordosis angle. Similarly, gauge 632 includes the letter H with an arrow indicating that this gauge represents an increase or decrease in height. Guage 632 also includes the letters mm, to indicate that the height numbers 556 are in millimeters.

Lordosis and height gauges 630, 632 may further comprise a mechanism (such as locking screws (not shown)) for locking sliders 642, 674 at a zero angle position on indicators 638, 648. Thus, the user may adjust sliders 642, 674 to ensure that they point to zero prior to adjustment of the height or angle of the implant. After the sliders have been “zeroed”, the locking screws may be tightened down to ensure that sliders 642, 674 do not move unless the adjustment devices displace them during adjustment of the height or lordosis angle of the implant.

Instrument 600 may further include a locking bar 660 and a cage locking screw 664 and a coupling mechanism 680, such as a bayonet or the like, for coupling and decoupling proximal handle 604 with shaft 602. These components are substantially similar in operation and function to similar components described above.

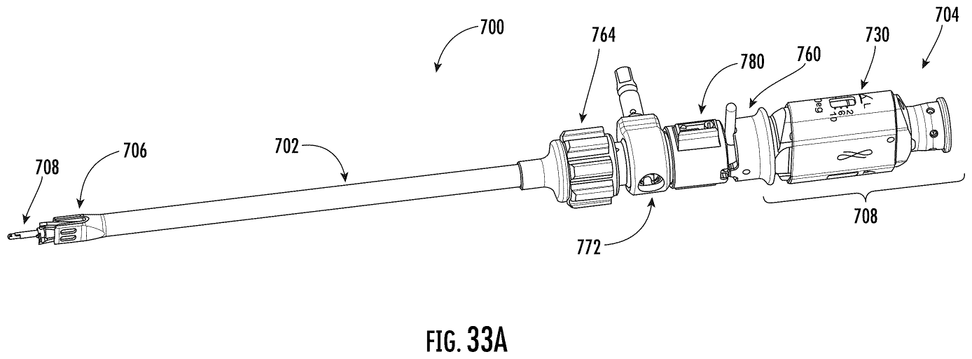

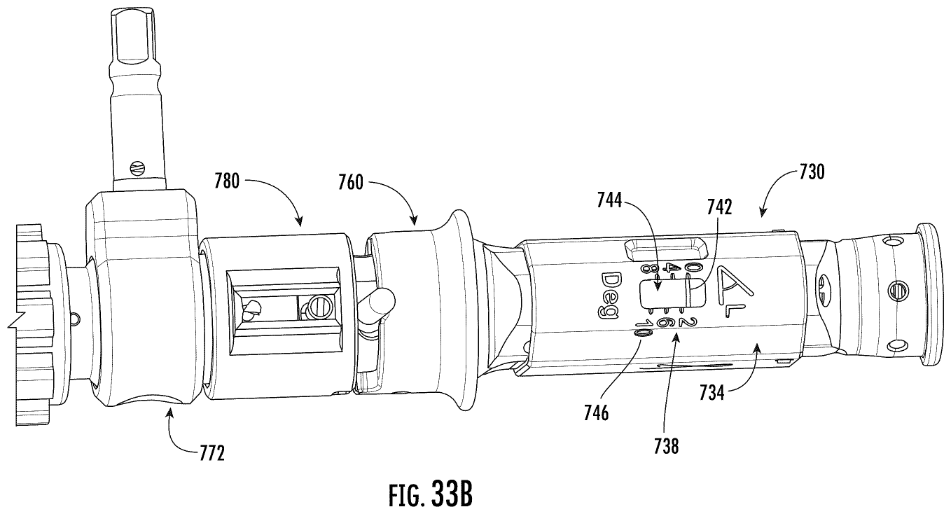

Referring now to FIGS. 33A and 33B, another embodiment of a deployment instrument 700 for use with the implants described above comprises an elongated shaft 702 with a proximal handle 704 and a distal gripping element 706 for removably coupling instrument 700 to a spinal implant (not shown). Instrument 700 may be particularly useful for placement of an implant (not shown) from a posterior approach outside of the facet joint (transforaminal lumbar interbody fusion or TLIF), such as the implant described in PCT/EP2022/069886, previously incorporated herein by reference.

Similarly to previous embodiments, elongate shaft 702 includes an actuator rod (not shown) configured for advancement and retraction through an internal bore of the implant for cooperating with one or more translation members within the implant to cause longitudinal movement of the translation members to adjust the height and the lordosis angle of the implant. The actuator rods may each include a distal mating feature (not shown) for coupling to corresponding mating features within the implant (see FIG. 5C). A more complete description of these elements can be found in International Patent Application Nos. PCT/EP2024/051180 and PCT/EP2022/069886, previously incorporated herein by reference.

Instrument 700 further comprises a lordosis adjustment device for adjusting a lordosis angle of the implant. In embodiments, these devices are substantially similar in operation and function to devices 140, 150 described above. Instrument 700 further comprises an actuation unit 708 in handle 704 for transferring the rotation of the adjustment device to longitudinal translation of the actuator rod similar to previous embodiments. In some embodiments, actuation unit 708 comprises one or more linkages that couple the mating features of the adjustment device with its corresponding gauge to rotate the needle with the gauge, and one or more linkages that couple the gauge with the corresponding actuator rods. In other embodiments, the linkages are reversed such that the adjustment devices are coupled to the actuator rods and the rods are coupled to the gauge.

Actuation unit 708 further comprises an indicator or gauge 730 on handle 704 for providing a user with an indication of a lordosis angle of the implant. The adjustment devices are operable to adjust the indicators on gauge 730 to match the discrete angle adjustments of the implant.

As shown more clearly in FIG. 33B, gauge 730 comprises an annular or circular housing 734 that extends around a portion of proximal handle 604. Housing 734 includes an indicator 738 including numbers 745 that represent discrete steps of the lordosis adjustment. These numbers correspond with discrete distances moved by the translation members within the implant. For example, numbers 746 indicate the angle between the upper and lower endplates of the implant (e.g., from 0 to 10 degrees). Gauge 730 further comprises a slider 742 coupled to the adjustment device such that rotation of the handles of this device causes slider 742 to move longitudinally through a slot 744 in housing 734 to provide the user with a very clear visual indication of the discrete adjustments made to the implant. Gauge 730 may also include visual indicator, such as any suitable alphanumeric symbol, such as LORDOSIS, and/or they may be color-coded to match the colors on the shafts of the adjustment devices. In the exemplary embodiment, gauge 730 includes the words Deg and a picture of an angle with the subscript L, for lordosis angle.

Lordosis gauge 730 may further comprise a mechanism (such as locking screws (not shown)) for locking slider 742 at a zero angle position on indicator 738. Thus, the user may adjust slider 742 to ensure that they point to zero prior to adjustment of the angle of the implant. After the slider has been “zeroed”, the locking screws may be tightened down to ensure that slider 742 do not move unless the adjustment devices displace them during adjustment of the height or lordosis angle of the implant.

Instrument 700 may further include a locking bar 760 and a cage locking screw 764 and a coupling mechanism 780, such as a bayonet or the like, for coupling and decoupling proximal handle 704 with shaft 702. These components are substantially similar in operation and function to similar components described above.

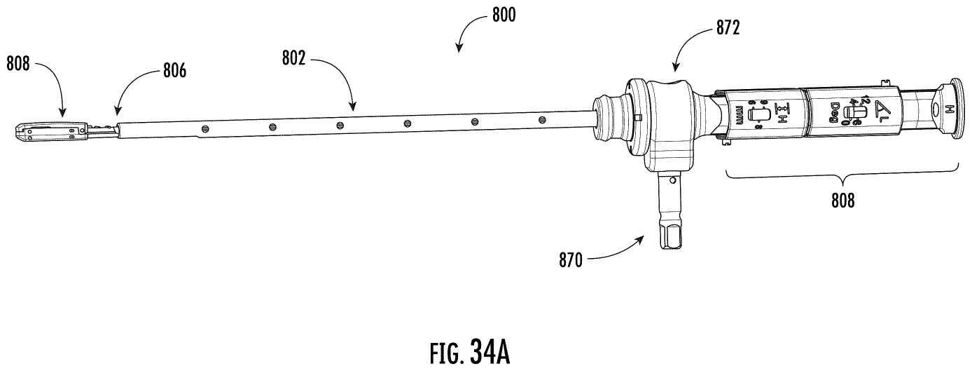

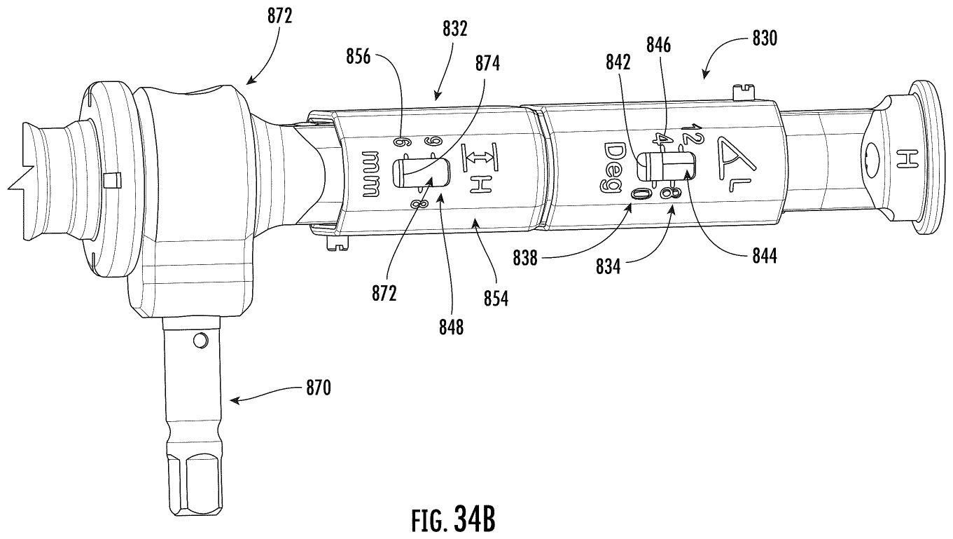

Referring now to FIGS. 34A and 34B, another embodiment of a deployment instrument 800 for use with the implants described above comprises an elongated shaft 802 with a proximal handle 804 and a distal gripping element 806 for removably coupling instrument 800 to a spinal implant (not shown). Instrument 800 may also be particularly useful for insertion of a trial device 820 from a posterior approach outside of the facet joint (transforaminal lumbar interbody fusion or TLIF) to optimize the size of the spinal implant.

Similarly to previous embodiments, elongate shaft 802 includes first and second actuator rods (not shown) configured for advancement and retraction through internal bores of the implant for cooperating with one or more translation members within the implant to cause longitudinal movement of the translation members to adjust the height and the lordosis angle of the implant. The actuator rods may each include a distal mating feature (not shown) for coupling to corresponding mating features within the implant (see FIG. 5C). A more complete description of these elements can be found in International Patent Application Nos. PCT/EP2024/051180 and PCT/EP2022/069886, which are incorporated herein by reference.

Instrument 800 further comprises a lordosis adjustment device for adjusting a lordosis angle of the implant and a height adjustment device for adjusting the overall height of the implant. In embodiments, these devices are substantially similar in operation and function to devices 140, 150 described above. Instrument 800 further comprises an actuation unit 808 in handle 804 for transferring the rotation of the adjustment devices to longitudinal translation of the actuator rods similar to previous embodiments. In some embodiments, actuation unit 808 comprises one or more linkages that couple the mating features of the adjustment devices with their corresponding gauges to rotate the needle with the gauges, and one or more linkages that couple the gauges with the corresponding actuator rods. In other embodiments, the linkages are reversed such that the adjustment devices are coupled to the actuator rods and the rods are coupled to the gauge. In yet another embodiment, actuation unit 808 comprises a single linkage that couples each of the mating features to both of the corresponding gauges and actuator rods.

Actuation unit 808 further comprises a first indicator or gauge 830 on handle 804 for providing a user with an indication of a lordosis angle of the implant, and a second indicator or gauge 832 on handle 804 for providing a user an indication of a height of the implant (discussed in more detail below). The adjustment devices are operable to adjust the indicators on gauge 830, 832, respectively, to match the discrete angle and height adjustments of the implant.

As shown more clearly in FIG. 34B, each gauge 830, 832 comprises an annular or circular housing 834, 854 that extends around a portion of proximal handle 804. Housings 834, 854 each include an indicator 838, 848 including numbers 846, 856 that represent discrete steps of height or lordosis adjustment. These numbers correspond with discrete distances moved by the translation members within the implant. For example, numbers 846 indicate the angle between the upper and lower endplates of the implant (e.g., from 0 to 12 degrees) and numbers 856 indicate the height or distance between the endplates (e.g., from 6 to 9 mm). Gauges 830, 832 further comprise a slider 842, 874 coupled to the adjustment devices such that rotation of the handles of these devices causes slider to move longitudinally through a slot 844, 872 in housings 834, 854 to provide the user with a very clear visual indication of the discrete adjustments made to the implant. Gauges 830, 832 may also include a visual indicator of each gauge (i.e., height or lordosis). These indicators may comprise any suitable alphanumeric symbol, such as LORDOSIS or HEIGHT, and/or they may be color-coded to match the colors on the shafts of the adjustment devices. In the exemplary embodiment, gauge 830 includes the words Deg and a picture of an angle with the subscript L, for lordosis angle. Similarly, gauge 832 includes the letter H with an arrow indicating that this gauge represents an increase or decrease in height. Guage 832 also includes the letters mm, to indicate that the height numbers 856 are in millimeters.

Lordosis and height gauges 830, 832 may further comprise a mechanism (such as locking screws (not shown)) for locking sliders 842, 874 at a zero angle position on indicators 838, 848. Thus, the user may adjust sliders 842, 874 to ensure that they point to zero prior to adjustment of the height or angle of the implant. After the sliders have been “zeroed”, the locking screws may be tightened down to ensure that sliders 842, 874 do not move unless the adjustment devices displace them during adjustment of the height or lordosis angle of the implant.

Instrument 800 may further include a locking bar 860 and a cage locking screw 864 and a coupling mechanism 880, such as a bayonet or the like, for coupling and decoupling proximal handle 804 with shaft 802. These components are substantially similar in operation and function to similar components described above. Instrument 800 may further include a lateral handle (not shown) coupled to a lateral extension 872 on proximal handle 804 of instrument 800. The lateral handle provides a gripping element for the surgeon to handle and control instrument 800.

Other embodiments will be apparent to those skilled in the art from consideration of the specification and practice of the embodiment disclosed herein. It is intended that the specification and examples be considered as exemplary only, with a true scope and spirit of the embodiment being indicated by the following claims.

For example, in accordance with a first aspect of this disclosure, a first embodiment of a surgical instrument is provided for inserting a medical device between adjacent vertebral bodies in a patient. The instrument comprises: an elongate shaft having a proximal handle and a distal end portion; a first mating feature for securing the distal end portion of the shaft to the medical device; a second mating feature for engaging a translation member within the medical device; and an adjustment device for moving the translation member to adjust an angle between upper and lower endplates of the medical device.

A second embodiment is the first embodiment of the surgical instrument, further comprising an actuator rod extending through the shaft and coupling the adjustment device with the second mating feature.

A third embodiment is any combination of the first two embodiments, wherein the adjustment device comprises a rotatable handle and a shaft, wherein the shaft is configured for insertion into a proximal opening on the rotatable handle.

A 4^{th} embodiment is any combination of the above embodiments, wherein rotation of the shaft of the adjustment device causes longitudinal translation of the actuator rod.

A 5^{th} embodiment is any combination of the above embodiments, wherein longitudinal translation of the actuator rod adjusts a distance between the distal ends of the upper and lower endplates.

A 6^{th} embodiment is any combination of the above embodiments, wherein the device comprises a longitudinal axis and the upper and lower endplates comprise first and second sides disposed on either side of the longitudinal axis, wherein longitudinal translation of the actuator rod adjusts a distance between the first sides of the upper and lower endplates.

A 7^{th} embodiment is any combination of the above embodiments, wherein proximal translation of the actuator rod causes the distance to increase.

An 8^{th} embodiment is any combination of the above embodiments, further comprising a gauge on the proximal handle configured to indicate said distance between the distal ends of the upper and lower endplates.

A 9^{th} embodiment is any combination of the above embodiments, wherein the gauge comprises a needle and is coupled to the shaft of the adjustment device and wherein rotation of said shaft causes rotation of the needle.

A 10^{th} embodiment is any combination of the above embodiments, wherein the distance between the distal ends of the upper and lower endplates is adjusted in discrete steps.

An 11^{th} embodiment is any combination of the above embodiments, wherein the gauge further comprises an indicator corresponding to the discrete steps.

A 12^{th} embodiment is any combination of the above embodiments, further comprising a locking mechanism on the proximal handle, wherein the locking mechanism is configured to prevent the shaft of the adjustment device from coupling to the proximal handle.

A 13^{th} embodiment is any combination of the above embodiments, wherein the locking mechanism comprises a rotatable knob on the proximal handle of the instrument.

A 14^{th} embodiment is any combination of the above embodiments, further comprising: a third mating feature on the distal end portion for engaging a second translation member within the medical device; and a second adjustment device for moving the second translation member to adjust a distance between the upper and lower endplates of the medical device.

A 15^{th} embodiment is any combination of the above embodiments, further comprising a second actuator rod extending through the shaft coupling the second adjustment device with the third mating feature.

A 16^{th} embodiment is any combination of the above embodiments, wherein the second adjustment device comprises a rotatable handle and a shaft, wherein the shaft is configured for insertion into a second proximal opening on the rotatable handle of the second adjustment device.

A 17^{th} embodiment is any combination of the above embodiments, wherein rotation of the shaft of the second adjustment device causes longitudinal translation of the second actuator rod.

An 18^{th} embodiment is any combination of the above embodiments, wherein longitudinal translation of the second actuator rod adjusts a distance between the distal and proximal ends of the upper and lower endplates.

A 19^{th} embodiment is any combination of the above embodiments, wherein proximal translation of the second actuator rod causes the distance to increase.

A 20^{th} embodiment is any combination of the above embodiments, further comprising a second gauge on the proximal handle configured to indicate the distance between the distal and proximal ends of the upper and lower endplates.

A 21^{st} embodiment is any combination of the above embodiments, wherein the second gauge comprises a needle and is coupled to the shaft of the second adjustment device and wherein rotation of said shaft causes rotation of the second needle.

A 22^{nd} embodiment is any combination of the above embodiments, wherein the distance between the distal and proximal ends of the upper and lower endplates is adjusted in discrete steps.

A 23^{rd} embodiment is any combination of the above embodiments, wherein the second gauge further comprises an indicator corresponding to the discrete steps.

A 24^{th} embodiment is any combination of the above embodiments, wherein the first mating feature comprises first and second gripping arms configured to engage and secure to a proximal portion of the medical device.

A 25^{th} embodiment is any combination of the above embodiments, further comprising an interface on the proximal handle coupled to the gripping arms, and configured to move the gripping arms laterally towards and away from each other.

In accordance with another aspect of this disclosure, a first embodiment of a system is provided. The system comprises: a medical device configured for insertion between adjacent vertebral bodies, the medical device comprising upper and lower endplates, and a translation member movably disposed between the upper and lower endplates; and an instrument comprising: an elongate shaft having a proximal handle and a distal end portion; a first mating feature on the distal end portion for engaging the translation member; and an adjustment device for moving the translation member to adjust an angle between upper and lower endplates of the medical device.

A second embodiment is the first embodiment of the system, wherein the medical device comprises a trial device.

A third embodiment is any combination of the above embodiments, wherein the medical device comprises a spinal implant.

A 4^{th} embodiment is any combination of the above embodiments, further comprising an actuator rod extending through the shaft and coupling the adjustment device with the first mating feature.

A 5^{th} embodiment is any combination of the above embodiments, wherein the adjustment device comprises a rotatable handle and a shaft, wherein the shaft is configured for insertion into a proximal opening on the rotatable handle.

A 6^{th} embodiment is any combination of the above embodiments, wherein rotation of the shaft of the adjustment device causes longitudinal translation of the actuator rod.

A 7^{th} embodiment is any combination of the above embodiments, wherein longitudinal translation of the actuator rod adjusts a distance between the distal ends of the upper and lower endplates.

An 8^{th} embodiment is any combination of the above embodiments, wherein the device comprises a longitudinal axis and the upper and lower endplates comprise first and second sides disposed on either side of the longitudinal axis, and wherein longitudinal translation of the actuator rod adjusts a distance between the first sides of the upper and lower endplates.

A 9^{th} embodiment is any combination of the above embodiments, wherein proximal translation of the actuator rod causes the distance to increase.

A 10^{th} embodiment is any combination of the above embodiments, wherein the instrument further comprises a gauge on the proximal handle configured to indicate the distance between the distal ends of the upper and lower endplates.

An 11^{th} embodiment is any combination of the above embodiments, wherein the gauge comprises a needle and is coupled to the shaft of the adjustment device and wherein rotation of said shaft causes rotation of the needle.

A 12^{th} embodiment is any combination of the above embodiments, wherein the distance between the distal ends of the upper and lower endplates is adjusted in discrete steps.

A 13^{th} embodiment is any combination of the above embodiments, wherein the gauge further comprises an indicator corresponding to the discrete steps.

A 14^{th} embodiment is any combination of the above embodiments, wherein the instrument further comprises a locking mechanism on the proximal handle, wherein the locking mechanism is configured to prevent the shaft of the adjustment device from coupling to the proximal handle.

A 15^{th} embodiment is any combination of the above embodiments, wherein the locking mechanism comprises a rotatable knob on the proximal handle of the instrument.

A 16^{th} embodiment is any combination of the above embodiments, wherein the device further comprises a second translation member disposed between the upper and lower endplates, and the instrument further comprises: a second mating feature on the distal end portion for engaging the second translation member; and a second adjustment device for moving the second translation member to adjust a distance between upper and lower endplates of the medical device.

A 17^{th} embodiment is any combination of the above embodiments, further comprising a second actuator rod extending through the shaft coupling the second adjustment device with the second mating feature.

A 18^{th} embodiment is any combination of the above embodiments, wherein the second adjustment device comprises a rotatable handle and a shaft, and wherein the shaft is configured for insertion into a second proximal opening on the rotatable handle of the second adjustment device.

A 19^{th} embodiment is any combination of the above embodiments, wherein rotation of the shaft of the second adjustment device causes longitudinal translation of the second actuator rod.

A 20^{th} embodiment is any combination of the above embodiments, wherein longitudinal translation of the second actuator rod adjusts a distance between the distal and proximal ends of the upper and lower endplates.

A 21^{st} embodiment is any combination of the above embodiments, wherein the instrument further comprises a second gauge on the proximal handle configured to indicate the distance between the distal and proximal ends of the upper and lower endplates.

A 22^{nd} embodiment is any combination of the above embodiments, wherein the second gauge comprises a needle and is coupled to the shaft of the second adjustment device and wherein rotation of said shaft causes rotation of the second needle.

A 23^{rd} embodiment is any combination of the above embodiments, wherein the distance between the distal and proximal ends of the upper and lower endplates is adjusted in discrete steps.

A 24^{th} embodiment is any combination of the above embodiments, wherein the second gauge further comprises an indicator corresponding to the discrete steps.

A 25^{th} embodiment is any combination of the above embodiments, wherein the instrument further comprises a third mating feature for securing the shaft of the instrument to the device.

A 26^{th} embodiment is any combination of the above embodiments, wherein the third mating feature comprises first and second gripping arms configured to engage and secure to a proximal portion of the upper and lower endplates of the device.

In still another aspect of this disclosure, a first embodiment of a surgical instrument for measuring a space between adjacent vertebral bodies in a patient is provided. The surgical instrument comprises: an elongate shaft having a proximal handle and a distal end portion; a first mating feature for securing the distal end portion of the shaft to a trial device; a second mating feature for engaging a translation member within the trial device; and an adjustment device for moving the translation member to adjust an angle between upper and lower endplates of the trial device.

A second embodiment is the first embodiment of the surgical instrument, further comprising an actuator rod extending through the shaft and coupling the adjustment device with the second mating feature.

A third embodiment is any combination of the above embodiments, wherein longitudinal translation of the actuator rod adjusts a distance between the distal ends of the upper and lower endplates.

A 4^{th} embodiment is any combination of the above embodiments, wherein the trial device comprises a longitudinal axis and the upper and lower endplates comprise first and second sides disposed on either side of the longitudinal axis, wherein longitudinal translation of the actuator rod adjusts a distance between the first sides of the upper and lower endplates.

A 5^{th} embodiment is any combination of the above embodiments, further comprising a gauge on the proximal handle configured to indicate said distance between the distal ends of the upper and lower endplates.

A 6^{th} embodiment is any combination of the above embodiments, further comprising: a third mating feature on the distal end portion for engaging a second translation member within the trial device; and a second adjustment device for moving the second translation member to adjust a distance between the upper and lower endplates of the trial device.

A 7^{th} embodiment is any combination of the above embodiments, further comprising a second gauge on the proximal handle configured to indicate the distance between the distal and proximal ends of the upper and lower endplates.

1. A surgical instrument for inserting a medical device between adjacent vertebral bodies, the instrument comprising:
an elongate shaft having a proximal handle and a distal end portion;
a first mating feature for securing the distal end portion of the elongate shaft to the medical device;
a second mating feature for engaging a translation member within the medical device; and
an adjustment device for moving the translation member to adjust an angle between upper and lower endplates of the medical device.

2. The surgical instrument of claim 1, further comprising an actuator rod extending through the shaft and coupling the adjustment device with the second mating feature.

3. The surgical instrument of claim 2, wherein the adjustment device comprises a rotatable handle and a shaft, wherein the shaft is configured for insertion into a proximal opening on the rotatable handle.

4. The surgical instrument of claim 3, wherein rotation of the elongate shaft of the adjustment device causes longitudinal translation of the actuator rod.

5. The surgical instrument of claim 4, wherein longitudinal translation of the actuator rod adjusts a distance between the distal ends of the upper and lower endplates.

6. The surgical instrument of claim 4, wherein the device comprises a longitudinal axis and the upper and lower endplates comprise first and second sides disposed on either side of the longitudinal axis, wherein longitudinal translation of the actuator rod adjusts a distance between the first sides of the upper and lower endplates.

7. The surgical instrument of claim 6, wherein proximal translation of the actuator rod causes the distance to increase.

8. The surgical instrument of claim 7, further comprising a gauge on the proximal handle configured to indicate said distance between the distal ends of the upper and lower endplates.

9. The surgical instrument of claim 8, wherein the gauge comprises a needle and is coupled to the shaft of the adjustment device, and wherein rotation of said shaft causes rotation of the needle.

10. The surgical instrument of claim 5, wherein the distance between the distal ends of the upper and lower endplates is adjusted in discrete steps, and wherein the gauge further comprises an indicator corresponding to the discrete steps.

11. The surgical instrument of claim 3, further comprising a locking mechanism on the proximal handle, wherein the locking mechanism is configured to prevent the shaft of the adjustment device from coupling to the proximal handle.

12. The surgical instrument of claim 1, further comprising:
a third mating feature on the distal end portion for engaging a second translation member within the medical device; and
a second adjustment device for moving the second translation member to adjust a distance between the upper and lower endplates of the medical device.

13. A system comprising:
a medical device configured for insertion between adjacent vertebral bodies, the medical device comprising upper and lower endplates, and a translation member movably disposed between the upper and lower endplates; and
an instrument comprising:
an elongate shaft having a proximal handle and a distal end portion;
a first mating feature on the distal end portion for engaging the translation member; and
an adjustment device for moving the translation member to adjust an angle between the upper and lower endplates of the medical device.

14. The system of claim 13, wherein the medical device comprises a trial device or a spinal implant.

15. The system of claim 13, wherein the instrument further comprises an actuator rod extending through the shaft and coupling the adjustment device with the first mating feature.

16. The system of claim 15, wherein the adjustment device comprises a rotatable handle and a shaft, wherein the shaft is configured for insertion into a proximal opening on the rotatable handle, and wherein rotation of the shaft of the adjustment device causes longitudinal translation of the actuator rod.

17. The system of claim 16, wherein longitudinal translation of the actuator rod adjusts a distance between the distal ends of the upper and lower endplates.

18. The system of claim 17, wherein the medical device comprises a longitudinal axis, and the upper and lower endplates comprise first and second sides disposed on either side of the longitudinal axis, wherein longitudinal translation of the actuator rod adjusts a distance between the first sides of the upper and lower endplates.

19. The system of claim 18, wherein the instrument further comprises a gauge on the proximal handle configured to indicate the distance between the distal ends of the upper and lower endplates, and wherein the gauge comprises a needle and is coupled to the shaft of the adjustment device, and wherein rotation of said shaft causes rotation of the needle.

20. The system of claim 19, wherein the instrument further comprises a locking mechanism on the proximal handle, wherein the locking mechanism is configured to prevent the shaft of the adjustment device from coupling to the proximal handle.
